(19) United States
(12) Patent Application Publication
(10) Pub. No.: US 2019/0169664 A1
(43) Pub. Date: Jun. 6, 2019

(54) MICROBIAL PLATFORM FOR PRODUCTION OF GLYCOSYLATED COMPOUNDS

(71) Applicant: UNIVERSITY OF GEORGIA RESEARCH FOUNDATION, INC., Athens, GA (US)

(72) Inventors: Yajun YAN, Bogart, GA (US); Yifei WU, Athens, GA (US); Xinxiao SUN, Beijing (CN); Yuheng LIN, Bogart, GA (US)

(73) Assignee: University of Georgia Research Foundation, Inc., Athens, GA (US)

(21) Appl. No.: 16/099,032

(22) PCT Filed: May 5, 2017
(86) PCT No.: PCT/US17/31326

§ 371 Date: Nov. 5, 2018

Related U.S. Application Data
(60) Provisional application No. 62/357,719, filed on Jul. 1, 2016.
(60) Provisional application No. 62/333,048, filed on May 6, 2016.

(51) Int. Cl. C12P 19/04; C12N 1/20; C12P 21/00; C12P 19/34

(52) U.S. Cl. CPC C12P 19/04; C12P 19/34; C12P 21/005; C12N 1/20

(57) ABSTRACT

Host cells are metabolically engineered to consume glucose and glycerol simultaneously, and to divert glucose from catabolic to anabolic pathways without adversely affecting glucose uptake.

This application claims the benefit of U.S. Provisional Applications Ser. No. 62/333,048, filed May 6, 2016, and Ser. No. 62/357,719, filed Jul. 1, 2016, each which is incorporated herein by reference in its entirety.

### BACKGROUND

Microorganisms utilize carbon sources such as glucose to grow, propagate, supply energy for various cellular processes, and generate biomolecules. In microorganisms, the catabolism of glucose is initially realized through glycolysis and pentose phosphate pathway (PPP) (Munoz-Elias et al., Cell. Microbiol. 8, 10-22 (2006)). These processes provide energy, reducing agents, and small molecules that promote glucose uptake, cell growth and other physiological activities. Anabolic activities utilizing glucose have been harnessed for microbial synthesis by metabolic engineering efforts. For instances, pyruvate, acetyl-CoA, and other small molecules derived from glucose catabolism can be converted or reassembled into fuels, bulk chemicals, fine chemicals, and even structurally complicated organic products through various biochemical reactions and biosynthetic mechanisms (Lin et al., Nat. Commun. 4, Article number: 2603 doi:10.1038/ncomms3603 (2013a); Lin et al., ACS Synth. Biol. 3, 497-505 (2014a); Lin et al., Metab. Eng. 23, 62-69 (2014b); Yuzawa et al., Biochemistry 51, 9779-9781 (2012); Sun et al., Appl. Environ. Microbiol. 79, 4024-4030 (2013); Lin et al. Metab. Eng. 18, 69-77 (2013b); Peralta-Yahya et al., Nature 488, 320-328 (2012); Santos et al., Metab. Eng. 13, 392-400 (2011); Atsumi et al., Nature 451, 86-U13 (2008); Stephanopoulos, Science 315, 801-804 (2007); Farmer et al., Nat. Biotechnol. 18, 533-537 (2000)). Catabolic processes, which lead to more carbon flux into biomass, reduce the utilization efficiency of glucose in anabolic processes.

### SUMMARY

Microbial host cells of the invention are metabolically engineered to divert glucose from catabolic to anabolic pathways in a manner that does not adversely affect glucose uptake. The engineered cells can simultaneously consume glucose and a secondary carbon source, such as glycerol, thereby facilitating the efficient conversion of C6 sugars into various glycosylated compounds of commercial and research interest.

In one aspect, the disclosure provides a genetically engineered microbe that includes at least one metabolic pathway modification that disrupts glucose catabolism. The metabolic pathway modification that disrupts glucose catabolism can include, without limitation, a modification of the glycolysis pathway, or a modification in the pentose phosphate pathway, or both. In some embodiments, the genetically engineered microbe optionally further includes at least one metabolic pathway modification that metabolically redirects phosphoenolpyruvate (PEP) for enhanced uptake of glucose. The metabolic pathway modification that metabolically redirects PEP can include, without limitation, a modification that disrupts a PEP-dependent glycerol assimilation pathway. In some embodiments, the genetically engineered microbe optionally further includes any one or more of a metabolic pathway modification that disrupts the conversion of UDP-glucose to UDP glucuronic acid, a metabolic pathway modification that eliminates the conversion of glucose-1-phosphate to glucolactone, a metabolic pathway modification that enhances the UDP-glucose biosynthetic pathway so as to direct more glucose into UDP-glucose, or any combination thereof.

In some embodiments, the genetically engineered microbe optionally further includes a metabolic pathway modification that enhances a biosynthetic pathway associated with the production of a glycosylated compound or a precursor of the glycosylated compound. The metabolic pathway modification that enhances a biosynthetic pathway associated with the production of a glycosylated compound or a precursor of the glycosylated compound can include, without limitation, a metabolic pathway modification that enhances the consumption or conversion of glucose-6-phosphate.

In some embodiments, the genetically engineered microbe optionally further includes a metabolic pathway modification that disrupts a metabolic pathway associated with degradation of a glycosylated compound, or a metabolic pathway that diverts a precursor away from the glycosylated compound, or both.

In some embodiments, the genetically engineered microbe simultaneously utilizes, as carbon sources, glucose and at least one secondary sugar. The secondary sugar can, without limitation, include glycerol, xylose, or any sugar or sugars extracted from, obtained from, or present in a lignocellulosic hydrolysate. In some embodiments of the genetically engineered microbe, the phosphoenolpyruvate (PEP) generated from consumption of the secondary sugar is utilized by the phosphotransferase system (PTS) so as to drive glucose uptake for production of a glycosylated compound.

In some embodiments of the genetically engineered microbe, a synergetic carbon utilization mechanism decouples glucose uptake from glucose catabolism by using glycerol as a carbon source to generate phosphoenolpyruvate (PEP) for operating the phosphotransferase system, or couples glucose uptake with glycerol catabolism via the phosphoenolpyruvate (PEP) as a driving force for glucose transport, or both.

An exemplary genetically engineered microbe includes at least one of the metabolic pathway modifications depicted in FIG. 1, Table 2, and/or Table 3; for example, it may include one, two, three, four, five, six, seven, eight, nine, ten, or more of the metabolic pathway modifications depicted in FIG. 1, Table 2, and/or Table 3. The genetically engineered microbe can, for example, include at least one of the following mutations, or combinations thereof: Δpgi, Δzwf, ΔpykA, ΔpykF, ΔgldA, Δugd, and Δgcd (E. coli) or their counterparts in other microbes. Optionally, the engineered microbe expresses or overexpresses at least one enzyme encoded by galU or pgm (E. coli) or counterparts in other microbes.

In an exemplary embodiment of the genetically engineered microbe, the microbe produces trehalose, and further comprises at least one of the following mutations, or combinations thereof: ΔtreA, ΔtreC, and ΔtreF (E. coli) or counterparts in other microbes.

In some embodiments, the genetically engineered microbe optionally expresses or overexpresses one or both enzymes encoded by otsA or otsB (E. coli) or counterparts in other microbes.

An exemplary genetically engineered microbe is an E. coli cell, or other microbe, that is engineered to include at least one of the following deletion mutations or sets of deletion mutations: ΔpgiΔzwf, ΔpykAFΔgldA; ΔtreACF; Δglk; ΔugdΔgcd; Δppc; or any combination thereof; or counterpart deletion mutations in other microbes. Optionally, the E. coli cell, or other genetically engineered microbe, can include least one plasmid expressing at least one enzyme operably encoded by at least one member of the group consisting of otsA, otsB, pgm, and galU, or counterparts in other genetically engineered microbes. Optionally, the E. coli cell, or other genetically engineered microbe, can be further metabolically engineered to enhance expression of phosphoglucomutase (pgm) or UTP-glucose-1-phosphate uridylyltransferase) (galU) or both.

The genetically engineered microbe can be a bacterial cell or a yeast cell. An exemplary bacterial cell is E. coli. Exemplary genetically engineered E. coli cells include, without limitation, E. coli cells represented by the strain designations, and characterized by the mutations present in said strains, as follows: YW-1, YW-2, YW-3, YW-3a, YW-3b, YW-4, YW-4Δlglk, YW-4b, YW-4bΔlglk, YW5, YW5b, YW6, YW6b, YW7, YW7b, and YW7c.

In another aspect, the disclosure provides a method for producing a glycosylated compound. A genetically engineered microbe, characterized by any feature or features described herein (e.g., mutations, deletions, metabolic pathway changes, overexpression of enzymes, etc.) or combination thereof, without limitation, can be cultured under conditions to produce the glycosylated compound. Exemplary glycosylated compounds that can be produced by the genetically engineered microbe include, without limitation, a glycoprotein, glycopeptide, glycolipid, proteoglycan, antibody, glycan, glycoside, polysaccharide, nucleotide and nucleic acid. In an exemplary embodiment, the genetically engineered microbe produces a polysaccharide, for example, trehalose, chondroitin or heparin. The glycosylated compound can be produced during a log phase of the microbial culture, or during a stationary phase of the microbial culture, or during both log and stationary phases. Optionally the glycosylated compound is isolated from the microbial culture, and optionally purified. In some embodiments of the method of producing a glycosylated compound, the microbial culture is supplied with glucose as well as a secondary carbons source, such as glycerol, xylose, or any sugar or sugars extracted from, obtained from, or present in a lignocellulosic hydrolysate.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the invention is not intended to describe each disclosed embodiment or every implementation of the invention. The description that follows more particularly exemplifies illustrative embodiments. In several places throughout the application, guidance may be provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic representation of synergetic carbon utilization mechanism and trehalose biosynthesis model in E. coli. Solid black arrows indicate native metabolic pathways in E. coli; broken black arrow indicates several steps in the pathway; thin black and white alternating dashed arrows on right side of figure indicate the trehalose biosynthesis model. Arrows with thick horizontal dashes indicate the critical blocked steps for the synergetic carbon utilization mechanism. Arrows having small gray dots indicate the main metabolic pathways of carbon sources in the synergetic carbon utilization mechanism. White arrow indicates the overexpression of the heterologous pathway from Lactococcus lactis. Gly, glycerol; Glc, glucose; G6P, glucose 6-phosphate; G1P, glucose 1-phosphate; UDPG, UDP-glucose; DHA, dihydroxyacetone; DHAP, glycerone phosphate; G3P, glycerol 3-phosphate; PEP, phosphoenolpyruvate; PYR, pyruvate; Tre6P, trehalose 6-phosphate; Tre, trehalose; OAA, oxaloacetate; PPP, pentose phosphate pathway; PTS, phosphotransferase system. pgi, encoding phosphoglucose isomerase (E.C. 5.3.1.9); zwf, encoding glucose 6-phosphate dehydrogenase (E.C. 1.1.1.49); pgm, encoding phosphoglucomutase (E.C. 5.4.2.2); galU, encoding glucose-1-phosphate uridylyltransferase (E.C. 2.7.7.9); glk, encoding glucokinase (E.C. 2.7.1.2); pykA, encoding pyruvate kinase II (E.C. 2.7.1.40); pykF, encoding pyruvate kinase I (E.C. 2.7.1.40); gldA, encoding glycerol dehydrogenase (E.C. 1.1.1.6); ppc, encoding phosphoenolpyruvate carboxylase (E.C. 4.1.1.31); ugd, encoding UDP-glucose 6-dehydrogenase (E.C. 1.1.1.22); gcd, encoding glucose dehydrogenase (E.C. 1.1.5.2); otsA, encoding trehalose-6-phosphate synthase (E.C. 2.4.1.15); otsB, encoding trehalose-6-phosphate phosphatase (E.C. 3.1.3.12); treA, encoding periplasmic trehalase (E.C. 3.2.1.28); treC, encoding trehalose-6-phosphate hydrolase (E.C. 3.2.1.93); treF, encoding cytoplasmic trehalase (E.C. 3.2.1.28); pyc, encoding pyruvate carboxylase (E.C. 6.4.1.1). See FIG. 1 in Wu et al., Metab. Eng., January 2017, 39:1-8, epub Nov. 3, 2016, for a colorized version of this drawing.

FIG. 2A-2C shows cell growth and consumption of carbon sources of YW-1. 2A) YW-1 was cultivated in M1 medium for 56 h; 2B) YW-1 was cultivated in M2 medium for 56 h; 2C) YW-1 were cultivated in M3 medium for 56 h. Wild type E. coli BW35113 was the control strain which was cultivated in the same condition as YW-1. WT: E. coli BW35113. The data were generated from three independent experiments. Error bars are defined as s.d.

FIG. 3 shows trehalose biosynthesis model construction in YW-3. The data were generated from three independent experiments. Error bars are defined as s.d.

FIG. 4A-4D shows cell growth and concentrations of trehalose and carbon sources of the engineered trehalose producing E. coli strains. 4A shows trehalose production and carbon source consumption of YW-3b cultivated in M4 medium; 4B shows trehalose production and carbon source consumption of YW-4b cultivated in M4 medium; 4C shows trehalose production and carbon source consumption of YW-6b cultivated in M4 medium; 4D shows trehalose production and carbon source consumption of YW-6b cultivated in M5 medium. The data were generated from three independent experiments. Error bars are defined as s.d.

FIG. 5 shows time courses of cell growth and consumption of carbon sources of YW-2. The data were generated from three independent experiments. Error bars are defined as s.d.

FIG. 6 shows detection of gluconeogenesis in YW-6b consuming 20 g l⁻¹ glycerol as sole carbon source. The data were generated from three independent experiments. Error bars are defined as s.d.

FIG. 7 shows detection of gluconeogenesis on YW-6b consuming 15 g l⁻¹ glycerol as sole carbon source. The data were generated from three independent experiments. Error bars are defined as s.d.

FIG. 8 shows growth and concentrations of trehalose and carbon sources for YW-7b and YW-7c cultivated in M4 medium in 48 h. The data were generated from three independent experiments. Error bars are defined as s.d.

### DESCRIPTION OF ILLUSTRATIVE EMBODIMENTS

We describe a microbial platform that is suitable for efficient glycosylation and biosynthesis of trehalose and other polysaccharides. With this technology, we can establish efficient microbial processes to convert C6 sugars into value-added polysaccharides and other glycosylated compounds, which will dramatically lower their production costs compared with the conventional chemical and biotechnological approaches. To demonstrate the applicability of this platform, high-level biosynthesis of trehalose was achieved. This disaccharide has a 25 million ton annual market.

Conventional glucose utilization in Escherichia coli depends on glycolysis and the pentose phosphate pathway to achieve catabolism. During this process, important catabolites such as acetyl-CoA and pyruvate contribute to cell growth and product synthesis. Unconventional utilization of glucose involves applying glucose as a C6 building block for production of glucose-based compounds. This non-catabolic usage of glucose conflicts with the catabolism which naturally leads to breakdown of glucose for biomass. If glucose is reserved as a building block by blocking catabolic pathways, cell growth is retarded, leading to lower productivity.

To address this conflict, we introduce a second carbon source glycerol and have designed a synergetic carbon utilization mechanism to strengthen the connection between glucose and glycerol utilization. This new mechanism couples glucose uptake and catabolism of glycerol via phosphoenolpyruvate (PEP) as a driving force for glucose transport.

As described in detail in Example I, we have validated the mechanism by introducing an exemplary glucose-based trehalose biosynthesis model. Before introducing the mechanism, the titer of trehalose is only 1.22 g l⁻¹ by consuming 7.39 g l⁻¹ glucose in 48 h. After enhancement and optimization of the mechanism, the titer of trehalose is 3.67 g l⁻¹ in 48 h by consuming 5.86 g l⁻¹ glucose. The conversion efficiency of glucose to trehalose is improved from 0.16 g trehalose/g glucose to 0.63 g trehalose/g glucose. After extension of cultivation time to 96 h, 8.20 g l⁻¹ trehalose is produced in shake flasks. Remarkably, the conversion efficiency of glucose to trehalose reaches 0.86 g trehalose/g glucose, which represents 91% of the theoretical maximum. This synergetic carbon utilization mechanism, which is established and demonstrated for the first time, can be applied for non-catabolic use of glucose as C6 building block for synthesis of glucose-based compounds. It also provides a novel strategy for industrial microbial production of trehalose.

The present invention provides a metabolically engineered microbial cell in which catabolism of glucose is diminished while anabolic processes involving glucose, such as glucose utilization as C6 building block or backbone precursor for glycosylation, are enhanced. Carbon flow within the cell is altered such that more carbon is directed toward production of useful products such as polysaccharides and glycosylated molecules. Microbial host cells are metabolically engineered to consume glucose and glycerol simultaneously, and to divert glucose from catabolic to anabolic pathways without adversely affecting glucose uptake. The metabolically engineered cells can advantageously be used to produce a wide variety of glucose-based products of commercial and research interest, including polysaccharides and other glycosylated compounds. The terms “glucose-based” and “glucose-derived” are used interchangeably herein and refer to a product with respect to which glucose is utilized as a C6 building block or backbone precursor during biosynthesis. Such a product may be referred to herein as, for instance, a “glucose-derived glycosylated compound” or a “glucose-derived product.”

Exemplary glycosylated compounds that can be produced by the metabolically engineered microbes of the invention (including “glucose-derived” products) include, without limitation, glycosylated biomolecules (also known as glycoconjugates) such as glycoproteins, glycopeptides, glycolipids, proteoglycans, antibodies, glycans, glycosides, polysaccharides, nucleotides and nucleic acids, and the like. Exemplary bacterial glycosides are reported in Elshahawi et al., 2015, Chem. Soc. Rev. 44(21), DOI: 10.1039/c4cs00426d. It should be noted that the term “glycosylated compound” as used herein is inclusive of polysaccharides. As used herein, the term polysaccharide includes a polysaccharide of any length, including a disaccharide, trisaccharide, tetrasaccharide, oligosaccharide, and higher level saccharide. A polysaccharide can be branched or unbranched. Disaccharides include reducing disaccharides, such as maltose and lactose, and nonreducing disaccharides, such as sucrose and trehalose. Additional reducing disaccharides include, without limitation, cellobiose, gentiobiose, isomaltose, laminarbiose, mannobiose and xylobiose. An exemplary oligosaccharide is raffinose; exemplary cyclic oligosaccharides include, α-, β-, and γ-cyclodextrins. Exemplary polysaccharides include, without limitation, amylopectin, amylose, cellulose, chitin, glycogen, chondroitin and heparin.

Catabolism of glucose can be diminished by attenuating or blocking either or both of glycolysis and the pentose phosphate pathway (PPP). However, making core metabolic changes such as disrupting the catabolism of glucose would typically be expected to have negative side effects, such as disrupting glucose uptake. Disruption of glucose uptake would be expected to adversely impact cellular metabolism or/or cell viability, which in turn would lead to low efficiency of glucose utilization as a C6 building block or backbone precursor. The metabolically engineered cells of the invention, however, are further engineered in a surprising and clever manner such that they continue to take up glucose even though catabolic processes are disrupted.

Moreover, it is well-known that in the presence of both glucose and a second carbon source, such as glycerol or xylose, microbial cells will typically exhaust glucose before taking up substantial quantities of the second carbon source. However the metabolically engineered microbial cells of the invention are advantageously engineered so as to utilize both glucose and a second carbon source simultaneously in a synergetic fashion that promotes continued glucose uptake, cell growth, and anabolic production of glucose-derived biomolecules.

The metabolically engineered cell of the invention is characterized by the disruption of catabolic utilization of glucose by blocking at least one of, and preferably both of, glycolysis and the pentose phosphate pathway (PPP) as well as, optionally, enhancement of the UDP-glucose biosynthetic pathway so as to direct more glucose into UDP-glucose. Optionally, the metabolically engineered cell of the invention further includes metabolic changes that disrupt the PEP-dependent glycerol assimilation pathway and/or eliminate the conversion of UDP-glucose to UDP glucuronic acid, and glucose-1-phosphate to glucolactone. The metabolically engineered cell of the invention is also preferably metabolically engineered to enhance the biosynthetic pathway(s) associated with the production of the glucose-derived product of interest.

The metabolically engineered cell of the invention can be described as synergetic. More particularly, the metabolic engineering employed within the cell can be used to achieve the synergetic and simultaneous utilization of multiple carbon sources, such as glycerol and glucose, increasing the utilization efficiency of both carbon sources. In some embodiments, the metabolic changes introduced into the cell advantageously permit phosphoenolpyruvate (PEP) generated from glycerol consumption to be coupled with the phosphotransferase system (PTS) so as to drive glucose uptake for subsequent use in glycosylation or polysaccharide production, even though glycolysis and the pentose phosphate pathway (PPP) are disrupted.

Catabolic utilization of glucose can be prevented by blocking glycolysis and/or the pentose phosphate pathway (PPP) to release carbon catabolite repression (CCR). E. coli can be metabolically engineered to block glycolysis and the pentose phosphate pathway by disrupting the genes encoding phosphoglucose isomerase (pgi) and glucose 6-phosphate dehydrogenase (zwf) yielding ΔpgiΔzwf. These mutations allow glycerol to be used as a carbon source, even in the presence of glucose. Glycerol can then be utilized for catabolic purposes. It is expected that the metabolically engineered cell of the invention will likewise be able to utilize other carbon sources, such as xylose, in the same synergetic manner as glycerol. The metabolically engineered cells of the invention are thus expected to be useful in consuming carbon sources obtained from natural products, such lignocellulosic hydrolysates.

Additionally or alternatively, the metabolically engineered cell can be engineered to disrupt PEP-dependent glycerol assimilation. This disruption can spare PEP so that it is available for phosphotransferase system (PTS) to use to enhance glucose uptake. Redirecting glycerol to generate more PEP for driving the phosphotransferase system is expected to increase glucose uptake. E. coli can be metabolically engineered to disrupt the PEP-dependent glycerol assimilation pathway by disrupting genes encoding pyruvate kinase I (pykF) and pyruvate kinase II (pykA) in the glycolysis pathway, and glycerol dehydrogenase (gldA) in the PEP-dependent glycerol assimilation pathway, yielding ΔpykAFΔgldA.

In some embodiments, disruption of glycolysis and/or the pentose phosphate pathway (exemplified by strain YW-1) coupled with disruption of PEP-dependent glycerol assimilation (exemplified by strain YW-2) may be sufficient to enhance production of the desired glucose-derived product. In other embodiments, even if catabolic utilization of glucose is prevented and PEP-dependent glycerol assimilation is disrupted, the cell may accumulate glucose 6-phosphate which will, in turn, inhibit glucose uptake into the cell. In that event, the microbial cell can be further metabolically engineered to enhance consumption or conversion of glucose-6-phosphate into certain end products. For example, an efficient pathway to direct glucose-6-phosphate toward use as a building block or backbone precursor for biosynthesis of a desired product (e.g., trehalose) can be introduced into the microbial cell, or an existing pathway can be enhanced. Additionally or alternatively, degradation pathways for the intended product can be disrupted (exemplified by strains YW-3 and YW-4); the microbial cell can be metabolically engineered to add or enhance a biosynthetic pathway for the product (exemplified by strain YW-3a); and/or the microbial cell can be metabolically engineered to strengthen pathways toward precursor (e.g., glucose-1-phosphate and/or UDP-glucose) of the product (exemplified by strains YW-3b and YW-4b). Additionally or alternatively, competing pathways that would otherwise divert the precursor(s) away from the desired product can be disrupted (exemplified by strains YW-5b and YW-6b)

In some embodiments, the metabolically engineered cell can be further engineered to disrupt the metabolic pathway involved in the consumption of UDP-glucose to UDP-glucuronic acid, and glucose-1-phosphate to glucolactone. E. coli can be metabolically engineered to disrupt this metabolic pathway by disrupting genes encoding UDP-glucose 6-dehydrogenase (ugd) and quinoprotein glucose dehydrogenase (gcd).

Additionally or alternatively, the metabolically engineered cell can be further engineered to express higher levels of enzymes intended to strengthen the UDP-glucose biosynthetic pathway so as to direct more glucose into UDP-glucose. To this end, E. coli can be metabolically engineered to enhance expression of phosphoglucomutase (pgm) and UTP-glucose-1-phosphate uridylyltransferase) (gall).

Some optional metabolic changes engineered into the microbial host cell are specific to the type of product that is desired to be produced. Those changes can include disrupting one or more degradation pathways and/or enhancing or introducing one or more biosynthetic pathways associated with the product. For example, if the desired product is trehalose, the microbial cell can be engineered to block one or more trehalose degradation pathways, and or express or overexpress one or more enzymes in a trehalose biosynthetic pathway. E. coli can be metabolically engineered to disrupt a trehalose degradation pathway by disrupting the genes encoding periplasmic trehalase (treA) and cytoplasmic trehalase (treF) and/or trehalose-6-phosphate hydrolase (treC). Additionally or alternatively, E. coli can be metabolically engineered to express or overexpress trehalose 6-phosphate synthase (otsA) and trehalose 6-phosphate phosphatase) (otsB).

The glucose-derived glycosylated compound produced by the metabolically engineered host cell can be a product that is naturally produced by the corresponding wild-type cell, or it can be a non-native product, such as a eukaryotic glycosylated biomolecule, that is not naturally produced by the corresponding wild-type microbial host cell. In the case of a non-native product, the microbial host cell is further engineered to include a metabolic pathway necessary for production of non-native product in the host cell. In some embodiments, a non-native metabolic pathway is introduced into the microbe in the form of one or more extrachromosomal vectors, such as plasmids. Optionally the microbial host cell can be further engineered to optimize the metabolic pathways involved in production of the non-native product, such as a glycosylated eukaryotic protein. See, e.g., Rosano et al., Front. Microbiol., 2015, 5:172; U.S. Pat. No. 8,999,668; Valderrama-Rincon et al., Nat. Chem. Biol., 2012 8(5):434-436.

### Microbial Host Cells

The microbial host cells are preferably yeast or bacterial cells, more preferably bacterial cells. E. coli is an exemplary illustrative organism for the production of glucose-derived products such as polysaccharides and other glycosylated biomolecules, but the invention is not intended to be limited to embodiments that utilize E. coli. Examples of microbial cells that can be engineered as described herein include, in addition to E. coli, a wide variety of bacteria and yeast including members of the genera Escherichia, Salmonella, Clostridium, Zymomonas, Pseudomonas, Bacillus, Rhodococcus, Alcaligenes, Klebsiella, Paenibacillus, Lactobacillus, Enterococcus, Arthrobacter, Brevibacterium, Corynebacterium Candida, Hansenula, Pichia and Saccharomyces. Particularly preferred hosts include: Escherichia coli, Bacillus subtilis Bacillus licheniformis, Alcaligenes eutrophus, Rhodococcus erythropolis, Paenibacillus macerans, Pseudomonas putida, Enterococcus faecium, Saccharomyces cerevisiae, Lactobacillus plantarum, Enterococcus gallinarium and Enterococcus faecalis. In preferred embodiments, the host cell is a bacterial cell, such as an E. coli or Streptomyces caeruleus cell. In a particularly preferred embodiment, the host cell of the present invention is an E. coli cell.

The terms “microbe” and “microbial cell” are used interchangeably with the term “microorganism” and mean any microscopic organism existing as a single cell (unicellular), cell clusters, or multicellular relatively complex organisms. Microorganisms include, for example, bacteria, fungi, algae, protozoa, microscopic plants such as green algae, and microscopic animals such as rotifers and planarians. Preferably, a microbial host used in the present invention is single-celled. Notwithstanding the above preferences for bacterial and/or microbial cells, it should be understood the metabolic pathway of the invention can be introduced without limitation into the cell of an animal, plant, insect, yeast, protozoan, bacterium, or archaebacterium.

A cell that has been genetically engineered to express one or more metabolic enzyme(s) and/or to disrupt expression of one or more metabolically active genes as described herein may be referred to as a “host” cell, a “recombinant” cell, a “metabolically engineered” cell, a “genetically engineered” cell or simply an “engineered” cell. These and similar terms are used interchangeably. A genetically engineered cell may contain one or more artificial sequences of nucleotides which have been created through standard molecular cloning techniques to bring together genetic material that is not natively found together. DNA sequences used in the construction of recombinant DNA molecules can originate from any species. For example, plant DNA may be joined to bacterial DNA, or human DNA may be joined with fungal DNA. Alternatively, DNA sequences that do not occur anywhere in nature may be created by the chemical synthesis of DNA, and incorporated into recombinant molecules. Proteins that result from the expression of recombinant DNA are often termed recombinant proteins. Examples of recombination are described in more detail below and may include inserting foreign polynucleotides (obtained from another species of cell) into a cell, inserting synthetic polynucleotides into a cell, or relocating or rearranging polynucleotides within a cell. Any form of recombination may be considered to be genetic engineering and therefore any recombinant cell may also be considered to be a genetically engineered cell.

Additionally or alternatively, a genetically engineered cell may contain one or more genetic mutations that alter, e.g., disrupt or enhance, at least one normal cellular activity. For example, a microbe that contains a gene knockout is a genetically engineered organism, even if it does not contain any artificial nucleotide sequences.

Genetically engineered cells are also referred to as “metabolically engineered” cells when the genetic engineering modifies or alters one or more particular metabolic pathways so as to cause a change in metabolism. The goal of metabolic engineering is to improve the rate and conversion of a substrate into a desired product. General laboratory methods for introducing and expressing or overexpressing native and nonnative proteins such as enzymes in many different cell types (including bacteria, plants, and animals) are routine and well known in the art; see, e.g., Sambrook et al, Molecular Cloning: A Laboratory Manual., Cold Spring Harbor Laboratory Press (1989), and Methods for General and Molecular Bacteriology, (eds. Gerhardt et al.) American Society for Microbiology, chapters 13-14 and 16-18 (1994). Metabolic pathway modifications can take any of a number of different forms. Metabolic pathway modifications include, without limitation, modifications that reduce, attenuate, disrupt, lessen, down regulate or eliminate, the expression of a metabolic enzyme, or the production of a metabolic precursor or intermediate; metabolic pathway mutations likewise include, without limitation, modifications that enhance, increase, or up regulate the expression of endogenous (native to the wild-type cell) or exogenous (not native to the wild-type cell) enzymes, or that introduce new (non-native) enzymes, including non-native biosynthetic pathways for metabolic precursors or intermediates, into the cell.

In some embodiments of the metabolically engineered cell of the invention, one or more genes encoding a metabolic enzyme are disrupted, for example, so as to divert the flow of carbon within the cell. Disruption of a gene can be accomplished by any convenient method known to one of skill in the art. For example, a gene can be completely knocked out, i.e., made inoperative, such that it does not express detectable amounts of the protein it encodes. Alternatively, expression of the gene can be reduced or attenuated such that a smaller amount of the encoded protein is expressed compared to the amount expressed in a comparable wild-type cell. Disruption of a gene can occur at the genomic level, for example, by mutating or deleting all or part of the nucleic acid sequence encoding the protein; it can occur at the level of transcription, such as by interfering with the production of mRNA; it can occur at the level of translation, such as by interfering with the production of a protein encoded by mRNA; or it can occur post-translationally, as by interference with the activity of the expressed protein through the action of an inhibitor, for example.

Additionally or alternatively, in some embodiments of the metabolically engineered cell of the invention, one or more biosynthetic pathways are introduced into the cell. The biosynthetic pathway can be one already native to the host cell, in which case expression of the endogenous enzyme will be enhanced. Alternatively, the biosynthetic pathway can represent a novel pathway not present in the native cell. The introduction of the biosynthetic pathway of the invention into a cell involves expression or overexpression of one or more enzymes included in the pathway. An enzyme is “overexpressed” in a recombinant cell when the enzyme is expressed at a level higher than the level at which it is expressed in a comparable wild-type cell. In cells that do not express a particular endogenous enzyme, or in cells in which the enzyme is not endogenous (i.e., the enzyme is not native to the cell), any level of expression of that enzyme in the cell is deemed an “overexpression” of that enzyme for purposes of the present invention.

As will be appreciated by a person of skill in the art, overexpression of an enzyme can be achieved through a number of molecular biology techniques. For example, overexpression can be achieved by introducing into the host cell one or more copies of a polynucleotide encoding the desired enzyme. The polynucleotide encoding the desired enzyme may be endogenous or heterologous to the host cell. Preferably, the polynucleotide is introduced into the cell using a vector; however, naked DNA may also be used. The polynucleotide may be circular or linear, single-stranded or double stranded, and can be DNA, RNA, or any modification or combination thereof. The vector can be any molecule that may be used as a vehicle to transfer genetic material into a cell. Examples of vectors include plasmids, viral vectors, cosmids, and artificial chromosomes, without limitation. Examples of molecular biology techniques used to transfer nucleotide sequences into a microorganism include, without limitation, transfection, electroporation, transduction, and transformation. These methods are well known in the art. Insertion of a vector into a target cell is usually called transformation for bacterial cells and transfection for eukaryotic cells, however insertion of a viral vector is often called transduction. The terms transformation, transfection, and transduction, for the purpose of the instant invention, are used interchangeably herein. A polynucleotide which has been transferred into a cell via the use of a vector is often referred to as a transgene.

Preferably, the vector is an expression vector. An “expression vector” or “expression construct” is any vector that is used to introduce a specific polynucleotide into a target cell such that once the expression vector is inside the cell, the protein that is encoded by the polynucleotide is produced by the cellular transcription and translation machinery. Typically an expression vector includes regulatory sequences operably linked to the polynucleotide encoding the desired enzyme. Regulatory sequences are common to the person of the skill in the art and may include for example, an origin of replication, a promoter sequence, and/or an enhancer sequence. The polynucleotide encoding the desired enzyme can exist extrachromosomally or can be integrated into the host cell chromosomal DNA. Extrachromosomal DNA may be contained in cytoplasmic organelles, such as mitochondria (in most eukaryotes), and in chloroplasts and plastids (in plants). More typically, extrachromosomal DNA is maintained within the vector on which it was introduced into the host cell. In many instances, it may be beneficial to select a high copy number vector in order to maximize the expression of the enzyme. Optionally, the vector may further contain a selectable marker. Certain selectable markers may be used to confirm that the vector is present within the target cell. Other selectable markers may be used to further confirm that the vector and/or transgene has integrated into the host cell chromosomal DNA. The use of selectable markers is common in the art and the skilled person would understand and appreciate the many uses of selectable markers.

Enzyme expression levels can be measured and compared by obtaining crude enzyme extracts from an engineered cell and a comparable wild-type cell, subjecting a suitable substrate to each enzyme extract, and measuring the amount of product. Common methods for measuring the amount of the product may include, without limitation, chromatographic techniques such as size exclusion chromatography, separation based on charge or hydrophobicity, ion exchange chromatography, affinity chromatography, or liquid chromatography. The genetically engineered cell of the invention will yield a greater activity than a wild-type cell in such an assay. Additionally, or alternatively, the amount of enzyme can be quantified and compared by obtaining protein extracts from the genetically engineered cell and a comparable wild-type cell and subjecting the extracts to any of number of protein quantification techniques which are well known in the art. Methods of protein quantification may include, without limitation, SDS-PAGE in combination with western blotting and mass spectrometry.

A gene encoding an enzyme may be obtained from a suitable biological source, such as a bacterial cell, using standard molecular cloning techniques. For example, genes may be isolated using polymerase chain reaction (PCR) using primers designed by standard primer design software which is commonly used in the art. The cloned sequences are easily ligated into any standard expression vector by the skilled person.

In one embodiment of the genetically engineered cell, separate, independent expression vectors are introduced into the host cell for each enzyme that is desired to be expressed (or overexpressed) within the host cell. When a single expression vector is used, each nucleotide sequence encoding a desired enzyme may be under the control of a single regulatory sequence or, alternatively, each nucleotide sequence encoding a desired enzyme may be under the control of independent regulatory sequences. In host cells that are metabolically engineered to enhance expression of an endogenous enzyme so as to increase the level of the endogenous enzyme in the host cell, the genetically engineered cell is optionally further engineered in modify the expression of the endogenous enzyme. For example, the regulatory sequences can be modified (e.g., introduction of stronger regulatory sequences having a higher affinity for the transcriptional machinery). Alternatively, gene sequences which increase the translation of the mRNA can be introduced (e.g., introduction of processing sequencing such as introns).

### Carbon Source

The metabolically engineered cells are able to utilize one or more secondary carbon sources in a synergetic manner that allows glucose to be diverted to anabolic or biosynthetic processes such as utilization in polysaccharide synthesis or glycosylation of compounds. Suitable second carbon sources include glycerol and/or xylose, although the cell can be engineered to utilize any desired carbon source. Gene expression in the various metabolic pathways involved in sugar utilization, as exemplified in the Table 1 below, can be enhanced, reduced or eliminated, as desired, to promote utilization of one or more sugars in addition to glucose. For example, a cell can be engineered to utilize one or more sugars present in a lignocellulosic hydrolysate or other biomass derived source of sugars.

TABLE 1
Examples of pathways for sugar utilization in bacteria

| | | | |
|---|---|---|---|
| Enzyme | Gene | Reaction catalyzed | Pathway |

Arabinose utilization

| | | | |
|---|---|---|---|
| Arabinose-Binding Protein | araF | Arabinose transport | Arabinose |
| | | | Uptake |
| Arabinose Transport Membrane | araH | Arabinose transport | Arabinose |
| Protein | | | Uptake |
| Arabinose ATPase Protein | araG | Arabinose transport | Arabinose |
| | | | Uptake |
| Arabinose Isomerase | araA | Arabinose → Ribulose | Arabinose |
| | | | Catabolism |
| Ribulokinase | araB | Ribulose → Ribulose-5-P | Arabinose |
| | | | Catabolism |

Galactose utilization

| | | | |
|---|---|---|---|
| Galactose Binding Protein | mglB | Galactose transport | Galactose |
| | | | Uptake |
| Galactose Transport Membrane | mglC | Galactose transport | Galactose |
| Protein | | | Uptake |
| Galactose ATPase Protein | mglA | Galactose transport | Galactose |
| | | | Uptake |
| Galactokinase | galK | Galactose → Galactose-1-P | Galactose |
| | | | Catabolism |

Glucose utilization

| | | | |
|---|---|---|---|
| Glucokinase | glk | Glucose → Glucose-6-P | Glucose |
| | | | Uptake |
| Glucosephosphotransferase | ptsG | Glucose → Glucose-6-P | Glucose |
| Enzyme II | | | Uptake |
| Mannose PTS Protein IIA(III) | manX | Glucose → Glucose-6-P | Glucose |
| | | | Uptake |
| Mannosephosphotransferase | manZ | Glucose → Glucose-6-P | Glucose |
| Enzyme IIB | | | Uptake |

Mannose utilization

| | | | |
|---|---|---|---|
| Mannose PTS Protein IIA(III) | manX | Mannose → Mannose-6-P | Mannose |
| | | | Uptake |
| Pel Protein | manY | Mannose → Mannose-6-P | Mannose |
| | | | Uptake |
| Mannosephosphotransferase | manZ | Mannose → Mannose-6-P | Mannose |
| Enzyme IIB | | | Uptake |

Xylose utilization

| | | | |
|---|---|---|---|
| Xylose Proton Symport Protein | xylE | Xylose transport | Xylose |
| | | | Uptake |
| Xylose Isomerase | xylA | Xylose → Xylulose | Xylose |
| | | | Catabolism |
| Xylulokinase | xylB | Xylulose → Xylulose-5-P | Xylose |
| | | | Catabolism |

### Production of the Glucose-Derived Product

Enhanced production of the disaccharide trehalose using metabolically engineered cells of the invention is shown in Example I. However, as noted elsewhere, the invention is by no means limited to production of trehalose as the glucose-derived product. For example, it is envisioned that other glucose-derived glycosylated compounds such as chondroitin and heparin can be readily produced by the metabolically engineered cells of the invention.

Additionally, it has been surprisingly discovered that glucose-derived products can be efficiently produced by the metabolically engineered cell of the invention in the stationary phase of microbial cell growth, not just in the log phase. In some embodiments, the synergetic efficiency between glucose and the second carbon source, such as glycerol, is even higher during the stationary phase because of the reduced carbon conversion into cell biomass, which is a highly desired feature in large-scale production. It was surprisingly found that PEP can still be generated from glycerol to drive glucose uptake even when cell growth stops.

Although the invention is described, as proof of principle, using E. coli as the host microbial cell for the metabolic engineering, analogous changes in the expression levels of analogous genes or in the expression levels of analogous proteins, native or exogenous, would be understood by one of skill in the art to result in analogous results as manifested in the increased production of carbon-derived biomolecules of interest.

### EXAMPLES

### Example I. Establishing a Synergetic Carbon Utilization Mechanism for Non-Catabolic Use of Glucose in Microbial Synthesis

Microorganisms utilize simple carbon sources such as glucose to propagate and generate molecules that are essential to life, which forms the foundation of the fermentation industry. In microorganisms, the catabolism of glucose is initially realized through glycolysis and pentose phosphate pathway (PPP) (Munoz-Elias et al., Cell. Microbiol. 8, 10-22 (2006)) which not only provides energy, reducing agents, and small molecules for continuous glucose uptake, cell growth and other physiological behaviors but also supports anabolic activities. Such activities have been greatly harnessed for microbial synthesis by metabolic engineering efforts. For instances, pyruvate, acetyl-CoA, and other small molecules derived from glucose catabolism can be converted or reassembled into fuels, bulk chemicals, fine chemicals, and even structurally complicated organic products through various biochemical reactions and biosynthetic mechanisms (Lin et al., Nat. Commun. 4, Article number: 2603 doi:10.1038/ncomms3603 (2013a); Lin et al., ACS Synth. Biol. 3, 497-505 (2014a); Lin et al., Metab. Eng. 23, 62-69 (2014b); Yuzawa et al., Biochemistry 51, 9779-9781 (2012); Sun et al., Appl. Environ. Microbiol. 79, 4024-4030 (2013); Lin et al. Metab. Eng. 18, 69-77 (2013b); Peralta-Yahya et al., Nature 488, 320-328 (2012); Santos et al., Metab. Eng. 13, 392-400 (2011); Atsumi et al., Nature 451, 86-U13 (2008); Stephanopoulos, Science 315, 801-804 (2007); Farmer et al., Nat. Biotechnol. 18, 533-537 (2000)). In addition to the above conventional utilization of glucose, non-catabolic use of glucose as C6 building block or backbone precursor for biosynthesis such as glycosylation and polysaccharide synthesis is also meaningful to microorganisms and critical for microbial synthesis. For example, glycosylation of natural products such as anthocyanin and puerarin, which is difficult to achieve through chemical synthesis, can greatly enhance their stability, bio-solubility and bioavailability (Lim et al., Appl. Environ. Microbiol. 81, 6276-6284 (2015); Wang et al., Enzyme Microb. Technol. 57, 42-47 (2014); Yan et al., Appl. Environ. Microbiol. 71, 3617-3623 (2005)). During these processes, glucose usually needs to be activated into UDP-sugars to provide intact glycosyl groups, which poses a real conflict to its regular catabolism and creates a dilemma to engineering such biosynthesis. Catabolism would dominate glucose utilization and lead more carbon flux into biomass, which would dramatically reduce the utilization efficiency of glucose as C6 building block or backbone precursor. However, reducing or eliminating such catabolism competition by attenuating or blocking the glycolysis and PPP would disrupt glucose uptake and cellular metabolism or affect cell viability, which would also lead to low efficiency of glucose utilization as C6 building block or backbone precursor for microbial synthesis.

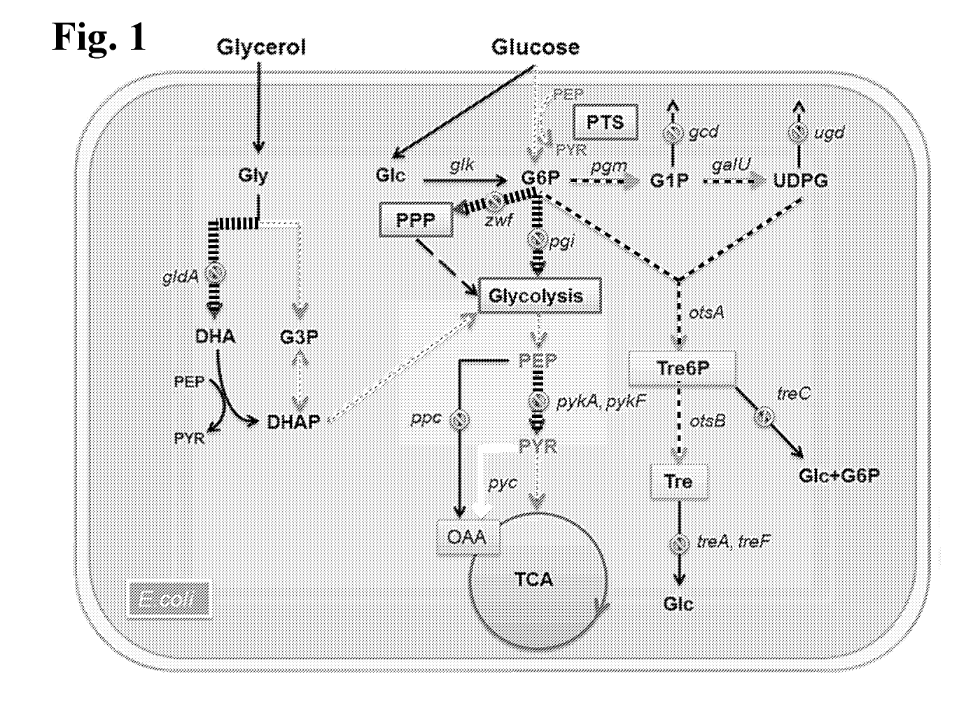

To address this dilemma, pioneering explorations have been attempted recently (Shiue et al., Biotechnol. Bioeng. 112, 579-587 (2015); Pandey et al., Appl. Microbiol. Biotechnol. 97, 1889-1901 (2013)). To solve the growth problem associated with blocking glycolysis and PPP, a second carbon source or enriched medium was used to support or recover cell growth. Although these efforts reserved glucose as building block or backbone precursor, the crosstalk or coupling between carbon sources has not been established and strengthened. The second carbon source was simply used for cell growth and had limited direct contribution to product formation, which led to low carbon conversion efficiency. To address this problem, we design a synergetic carbon utilization mechanism by utilizing glycerol as the second carbon source. More specifically, we introduce glycerol and rationally modify its assimilation to enhance the driving force PEP for glucose transport into cells and subsequent utilization as building block. As rationale, glucose enters cells as glucose-6-phosphate mainly through the PEP-dependent phosphotransferase system (PTS) (Hernandez-Montalvo et al., Biotechnol. Bioeng. 83, 687-694 (2003)). The system connected with glycolysis and PPP realizes the regeneration of PEP to support continuous glucose uptake and to sustain cellular metabolism and cell growth (FIG. 1). When the glycolysis and PPP are blocked and PEP cannot be regenerated from glucose, directing glycerol assimilation to enhance PEP generation as the driving force for glucose uptake would achieve the synergetic unitization of both carbon sources and increase the utilization efficiency of both carbon sources.

To validate and optimize this mechanism and examine the applicability of this mechanism in microbial synthesis, we choose trehalose as the target product and establish a glucose-based trehalose biosynthesis model in Escherichia coli. Trehalose is a non-reducing disaccharide with very stable characteristics.

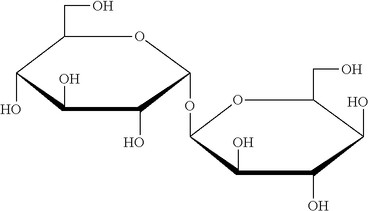

Trehalose has a wide range of applications in the food and pharmaceutical industries, due to its protective function on biological molecules under oxidative or extreme conditions (Schiraldi et al., Trends Biotechnol. 20, 420-425 (2002); Ohtake et al., J. Pharm. Sci. 100, 2020-2053 (2011); Kidd et al., Nat Biotech. 12, 1328-1329 (1994)). For instance, trehalose can be used to stabilize vaccines and preserve organs (Patist et al., Colloids Surf B Biointerfaces 40, 107-113 (2005); Kim et al., J. Control Release 142, 187-195 (2010)). Most recently, it has been reported that trehalose could find the applications in the treatment of fatty liver disease as well as diabetes and Alzheimer's disease by triggering autophagy (Torrice, Chem. Eng. News 94, 9-9 (2016)). Its annual market value was estimated to be 206.41 million US dollars in the year 2015 (Global Trehalose Market Size. (http://globalqyresearch.com/press-releases/global-trehalose-market). Currently, its industrial production completely relies on the enzymatic conversion of starch or maltose, which still suffers from side product formation (Kobayashi et al., J. Ferment. Bioeng. 83, 296-298 (1997); Mukai et al., Starch-Starke 49, 26-30 (1997); Yoshida et al., Starch-Starke 49, 21-26 (1997); Koh et al., Biotechnol. Lett. 20, 757-761 (1998); Koh et al., Carbohydr. Res. 338, 1339-1343 (2003)). In this study, we first develop a glucose-based trehalose biosynthesis model in E. coli, leading to the production of 1.59 g/L trehalose within 48 h by consuming 7.68 g/L glucose in shake flasks. With validation and enhancement of the synergetic carbon utilization mechanism in the trehalose biosynthesis model, we achieve remarkable 8.20 g/L trehalose production in shake flasks with elongated cultivation time. Surprisingly, the conversion efficiency of glucose to trehalose reaches 0.86 g trehalose/g glucose, representing 91% of the theoretic maximum. We find that the gluconeogenesis from glycerol also slightly contributes to this high efficiency. Overall, our results suggest that the synergetic carbon utilization mechanism has general applicability in microbial synthesis involving glucose as C6 building block or backbone precursor. In addition, this study demonstrates a novel microbial approach for trehalose production and has great scale-up potential.

The presence of strong glucose catabolism and its indispensable association with continuous glucose uptake and cellular metabolism create a dilemma for non-catabolic use of glucose as C6 building block or backbone precursor for microbial synthesis. To address this dilemma, we design a synergetic carbon utilization mechanism, which decouples glucose uptake from its catabolism by using glycerol to generate the driving force PEP for running (i.e., for operating) the phosphotransferase system. As proof-of-concept, a glucose-based trehalose biosynthesis model is developed for validation and optimization of this mechanism, which leads to high-level production of 8.20 g/L trehalose in shake flasks. Remarkably, the conversion efficiency of glucose to trehalose reaches 91% of the theoretic maximum due to the slight contribution from gluconeogenesis. This work indicates this mechanism can be generally applied to microbial synthesis involving glucose as C6 building block or backbone precursor and demonstrates great scale-up potential as a novel microbial approach for trehalose production. See Wu et al., Metab. Eng., January 2017, 39:1-8, epub Nov. 3, 2016.

### Experimental Results

Design and Development the Synergetic Carbon Utilization Mechanism.

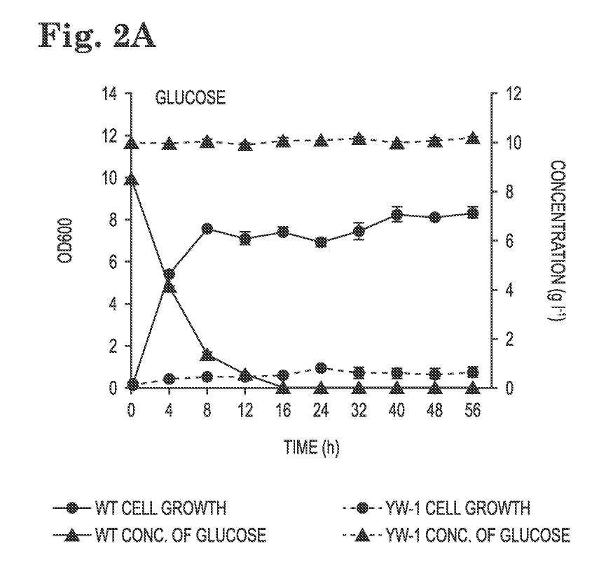

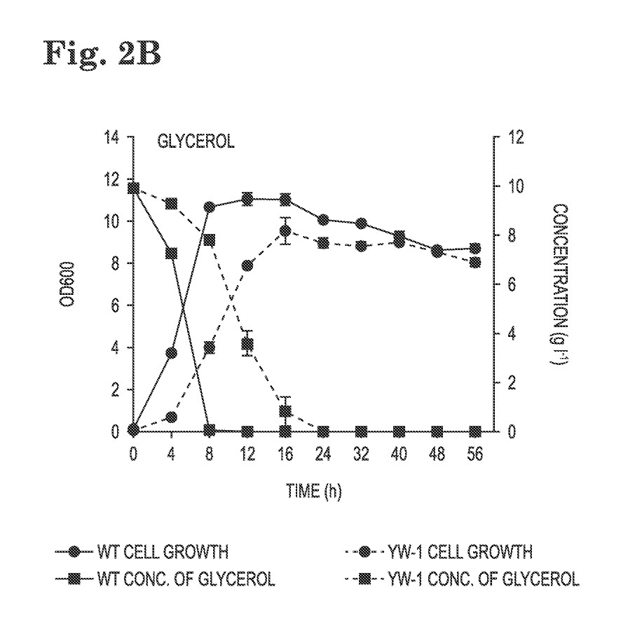

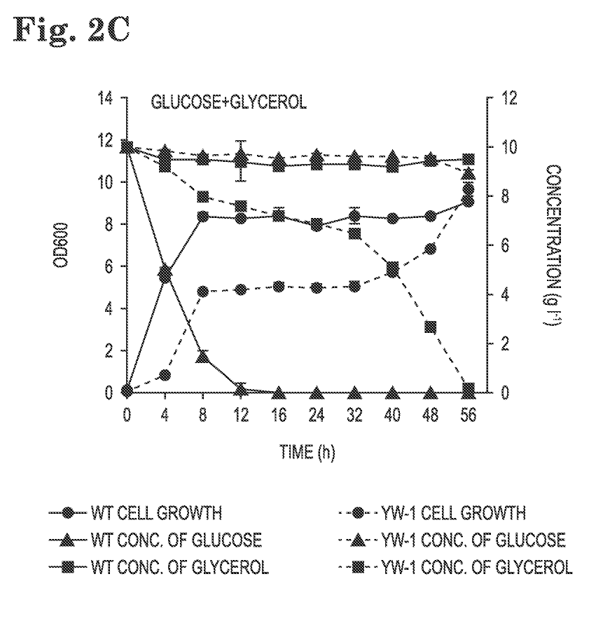

To efficiently use glucose as building block for microbial synthesis, we designed a synergetic utilization mechanism, which can reserve glucose for non-catabolic use and utilize PEP generated from a second carbon source to sustain glucose uptake. To examine this concept, we used glycerol as the second carbon source. Due to the carbon catabolite repression (CCR) in E. coli, glucose is preferentially used for cellular metabolism and cell growth when both glucose and glycerol are used as the carbon sources (Deutscher, Curr. Opin. Microbiol. 11, 87-93 (2008)). Hence, we deleted genes pgi (encoding phosphoglucose isomerase) and zwf (encoding glucose 6-phosphate dehydrogenase) in a wild type E. coli strain BW25113 to prevent the catabolic utilization of glucose by blocking both glycolysis and PPP and to release CCR. When we grew the resulting strain YW-1 in M1 medium that contains glucose as carbon source and a certain amount of yeast extract as additional nutrition, we observed that almost no glucose was consumed. The strain grew slightly (OD₆₀₀ value less than 1 in 56 h) due to the presence of yeast extract. The control strain BW25113 consumed glucose and grew normally with the OD₆₀₀ value over 8 in 56 h (FIG. 2A). In contrast, both YW-1 and the control strain BW25113 grew normally in M2 medium, in which glycerol was used to replace glucose as the carbon source. As show in FIG. 2B, glycerol was depleted in 24 hours by YW-1 and the OD600 value was able to reach around 10. Comparatively, the control strain BW25113 consumed glycerol a little bit faster and accumulated more biomass. Furthermore, when both glucose and glycerol were used as mixed carbon sources in M3 medium for cell growth, YW-1 consumed glycerol for cell growth and the OD₆₀₀ value was 9.67±0.31 in 56 h. The consumption of glucose was not obvious in 48 h and measured as 1.05±0.10 in 56 h. In contrast, the control strain BW25113 depleted glucose in 12 h and hardly consumed glycerol during the cell growth period due to CCR (FIG. 2C). These results indicated that blocking the glycolysis and PPP by deleting pgi and zwf was able to efficiently release the repression of glucose on glycerol utilization. However, in the present of both glucose and glycerol we didn't observe obvious glucose uptake by YW-1, which was expected to be driven by the PEP generated from glycerol since the PEP-dependent PTS is still active and dominates glucose transport into cells (Hernandez-Montalvo et al., Biotechnol. Bioeng. 83, 687-694 (2003); Steinsiek et al., J. Bacteriol. 194, 5897-5908 (2012)). As our first hypothesis, we think this might be due to the insufficient PEP intracellular availability for running PTS during the native glycerol catabolism.

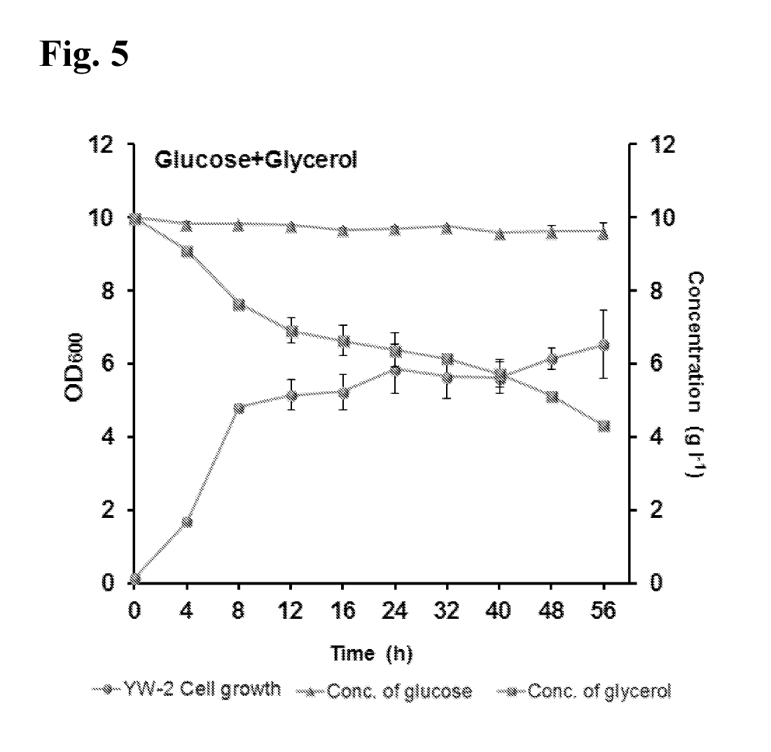

To examine this hypothesis, we further deleted pykF (encoding pyruvate kinase I) and pykA (encoding pyruvate kinase II) in the glycolysis and gldA (encoding glycerol dehydrogenase) in the PEP-dependent glycerol assimilation pathway (FIG. 1) in strain YW-1 to reserve PEP for PTS use. Interestingly, in M3 medium, the generated strain YW-2 didn't led to any increase in glucose uptake. The consumption of glucose (0.37±0.22 g/L) was even less than that of YW-1 (FIG. 5). Meanwhile, the cell biomass and consumption of glycerol were less than those of YW-1. To further address the glucose uptake issue in the synergetic mechanism, we reasoned that the lack of efficient pathways utilizing glucose as building block or backbone precursor for biosynthesis might lead to the accumulation of glucose 6-phophate and inhibit glucose uptake into cells (Morita et al., J. Biol. Chem. 278, 15608-15614 (2003)). To examine this hypothesis, releasing such inhibition by enhancing the consumption or conversion of glucose 6-phoshphate into certain end products could be an effective approach. As proof-of-concept, we chose trehalose as the target product to further validate the synergetic carbon utilization mechanism.

Construction of Glucose-Based Trehalose Biosynthesis Model.

E. coli BW25113 has its native trehalose biosynthetic pathway composed of osmatic inducible otsA (encoding trehalose 6-phosphate synthase) and otsB (encoding trehalose 6-phosphate phosphatase) (Kandror et al., Proc. Natl. Acad. Sci. USA 99, 9727-9732 (2002); Strom et al., Mol. Microbiol. 8, 205-210 (1993)). However, when we cultivated E. coli BW25113 in M9Y medium, no trehalose production was detected. We speculated that trehalose might be degraded and/or otsA and otsB might not be expressed. To verify it, we did feeding experiments using E. coli BW25113 with 5 g/L of trehalose.2H₂O in the medium. We observed that trehalose disappeared in 12 h, which indicated that the trehalose degradation or consumption was very strong in the wild type strain. As shown in FIG. 1, two enzymes encoded by treA (encoding periplasmic trehalase) and treF (encoding cytoplasmic trehalase) are responsible for trehalose degradation in E. coli (Strom et al., Mol. Microbiol. 8, 205-210 (1993)). To prevent trehalose degradation, we first did the same feeding experiments using two single-gene knockout strains BW25113/ΔtreA and BW25113/ΔtreF. The results showed that these two strains were also able to consume all the trehalose, which indicated that both enzymes were actively expressed and contributed to trehalose degradation in strain BW25113. Therefore, we deleted both treA and treF to block the degradation pathways in E. coli BW25113. In addition, to avoid the degradation of the intermediate trehalose 6-phosphate during trehalose biosynthesis, we further deleted gene treC (encoding trehalose-6-phosphate hydrolase) (FIG. 1), generating strain YW-3 (BW25113 ΔtreAΔtreCΔtreF). When we used strain YW-3 to do the feeding experiments, we did not observe any obvious trehalose degradation.

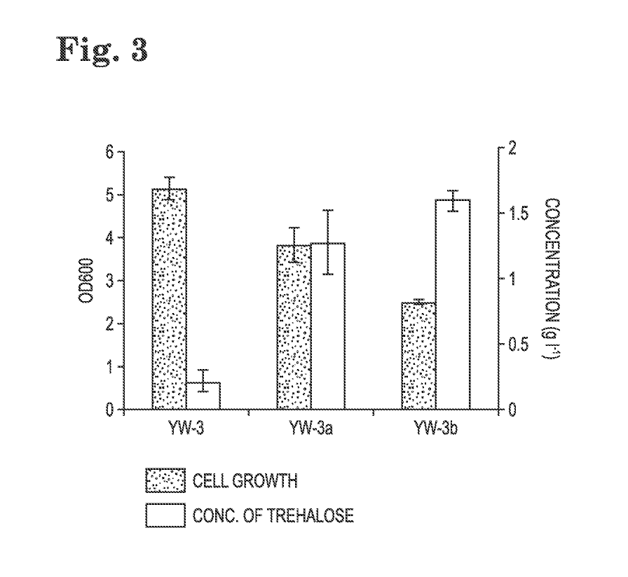

Interestingly, when we used M1 medium that contain glucose as the carbon source to cultivate YW-3, we detected that 0.21±0.07 g/L of trehalose was produced by YW-3 in 48 h by consuming 9.44±0.19 g/L glucose. The OD₆₀₀ value was around 5 (FIG. 3). This indicated that native expression of otsAB genes was weak and could lead to a small amount of trehalose production. To raise the trehalose productivity, we consecutively cloned otsA and otsB into plasmid pZE12-luc as an operon, generating pYW-1. As we expected, YW-3a that was generated by transferring pYW-1 into YW-3, produced 1.27±0.22 g/L of trehalose in M1 medium in 48 h by consuming 9.73±0.20 g/L glucose and the value of OD₆₀₀ decreased to 3.84±0.40. These results indicated that overexpression of trehalose biosynthetic pathway could direct more glucose from cell growth to trehalose production. Then we tried to strengthen the UDP-glucose biosynthetic pathway to direct more glucose into UDP-glucose which is one of the precursors of trehalose biosynthesis. For this purpose, we cloned pgm (encoding phosphoglucomutase) and galU (encoding UTP-glucose-1-phosphate uridylyltransferase) into vector pCS27 as an operon, generating pYW-2. Remarkably, YW-3b, which was generated by co-transferring pYW-1 and pYW-2 into YW-3, showed higher trehalose production. 1.59±0.07 g/L of trehalose was produced in 48 h by consuming 7.68±0.21 g/L glucose. The OD₆₀₀ value further dropped to 2.51±0.05. Compared with YW-3, it showed a 9.3-fold increase in the conversion efficiency of glucose to trehalose. In addition, from FIG. 3, we found that there was a negative correlation between cell growth and trehalose production with the enhancement of trehalose biosynthesis. These results also suggested the direct competition exists between glucose catabolism for cell growth and non-catabolic use of glucose for microbial synthesis. Therefore, the glucose-based trehalose biosynthesis demonstrated in YW-3b could serve as a good model for validating the synergetic carbon utilization mechanism.

Validation of the Synergetic Carbon Utilization Mechanism.

For the validation, we introduced the synergetic carbon utilization mechanism into the trehalose biosynthesis model by deleting pgi and zwf in YW-3. The generated strain YW-4 (BW25113 ΔtreAΔtreCΔtreFΔpgiΔzwf) demonstrated similar growth phenotype to YW-1 (BW25113 ΔpgiΔzwf) in M1 medium, which grew weakly and hardly consumed glucose. We further introduced pYW-1 and pYW-2 into YW-4, generating strain YW-4b, for trehalose biosynthesis. In M1 medium containing glucose as the carbon source, strain YW-4b only produced 0.24±0.01 g/L of trehalose in 48 h by consuming 0.47±0.18 g/L glucose. The OD₆₀₀ value was measured as around 0.45.

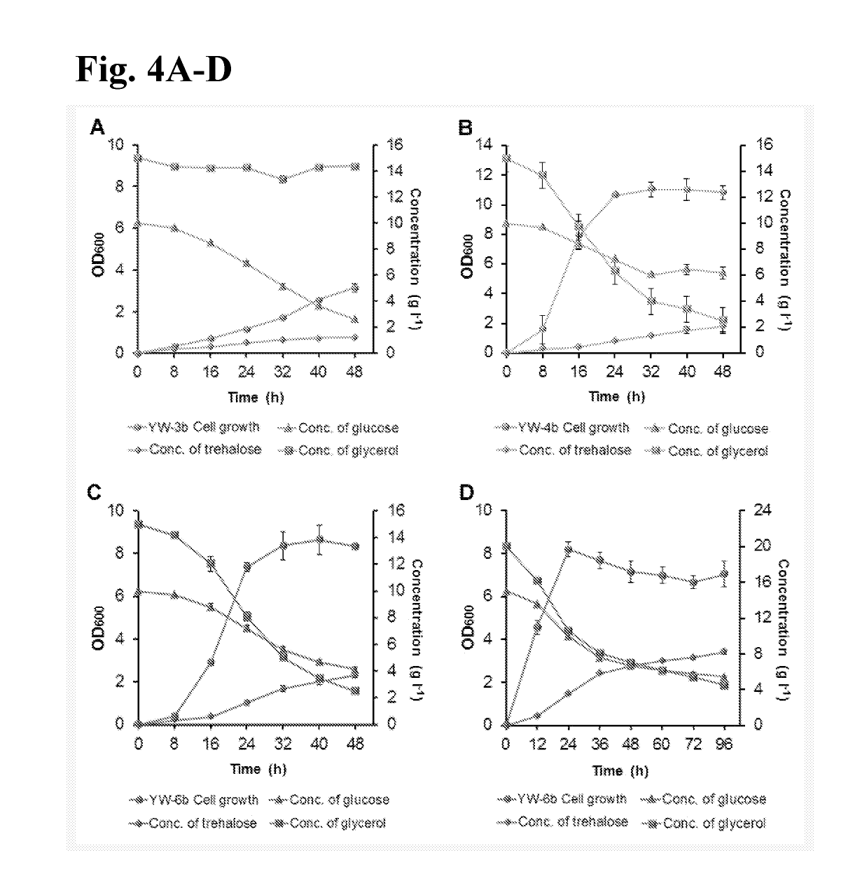

However, when M4 medium, to which glycerol was added as the second carbon source to generate PEP for glucose uptake, was used for cultivating YW-4b, 2.05±0.48 g/L of trehalose was produced in 48 h and glucose was consumed by 3.82±0.49 g/L. In the meantime, YW-4b consumed 12.51±0.88 g/L glycerol with an OD₆₀₀ value of 10.84±0.48 (FIG. 4b). As the control, strain YW-3b only produced 1.22±0.01 g/L trehalose by consuming 7.39±0.00 g/L glucose under the same condition. Nevertheless, YW-3b only consumed 0.55±0.03 g/L glycerol with an OD₆₀₀ value of 3.14±0.03 in 48 h (FIG. 4A). In summary, simply introducing the synergetic carbon utilization mechanism into the trehalose biosynthesis model was able to increase the conversion efficiency of glucose to trehalose by around 3.1 folds (from 0.17 to 0.537 g trehalose/g glucose). However, the utilization efficiency of glycerol was low (only at 0.16 g trehalose/g glycerol). These results suggested PEP generated from glycerol consumption could be coupled with PTS to drive glucose uptake when the glycolysis and PPP were disrupted. Furthermore, we deleted glk in strain YW-4 to test the effect of glucokinase on trehalose biosynthesis. For the glk-deficient strain harboring pYW-1 and pYW-2, the titer of trehalose and utilization of carbon sources were slightly lower than those of YW-4b, which indicated that the contribution of glucokinase to glucose uptake and trehalose biosynthesis was minor, which is consistent with the previous study showing that active PTS represses glucokinase and dominates glucose uptake.

To further prevent glucose loss during trehalose biosynthesis, we deleted two more genes ugd (encoding UDP-glucose 6-dehydrogenase) and gcd (encoding quinoprotein glucose dehydrogenase) in YW-4, generating YW-5, to eliminate the consumption of UDP-glucose to UDP-glucaronic acid and glucose 1-phosphate to glucolactone (FIG. 1). In M4 medium, YW-5b (YW-5 carrying pYW-1 and pYW-2) produced 2.43±0.08 g/L trehalose by consuming 4.47±0.20 g/L glucose in 48 h with an OD₆₀₀ value of 10.07±0.46. The titer of trehalose was 19% higher than that of YW-4b. In the meantime, the conversation efficiency of glucose to trehalose (0.544 g trehalose/g glucose) and the glycerol utilization efficiency (0.21 g trehalose/g glycerol) were all slightly higher than those of YW-4b. These results indicated that blocking competing pathways in trehalose biosynthesis could improve trehalose production. However, the glucose conversion efficiency and glycerol utilization efficiency were still not desirable. Hence, we further enhanced the synergetic carbon utilization mechanism on the basis of YW-5.

Enhancement of the Synergetic Carbon Utilization Mechanism.

We hypothesized that redirecting glycerol to generate more PEP for driving PTS would strengthen glucose uptake and enhance the synergetic carbon utilization mechanism. To examine this hypothesis, we deleted pykA, pykF, and gldA in YW-5 to reduce the utilization of PEP that is not associated with PTS (FIG. 1), generating strain YW-6. In M4 medium, YW-6 harboring pYW-1 and pYW-2 (YW-6b) was able to produce 3.67±0.22 g/L of trehalose in 48 h by consuming 5.86±0.20 g/L glucose and 12.48±0.14 g/L glycerol (FIG. 4C). The titer of trehalose increased by 51% compared with that of YW-5b. The conversion efficiency of glucose to trehalose and the glycerol utilization efficiency were further improved to 0.63 g trehalose/g glucose and 0.29 g trehalose/g glycerol. YW-6b had a lower OD₆₀₀ value (8.33±0.13) than that of YW-5b (10.07±0.46). These results indicated that improving PEP generation from glycerol could enhance the synergetic carbon utilization mechanism.

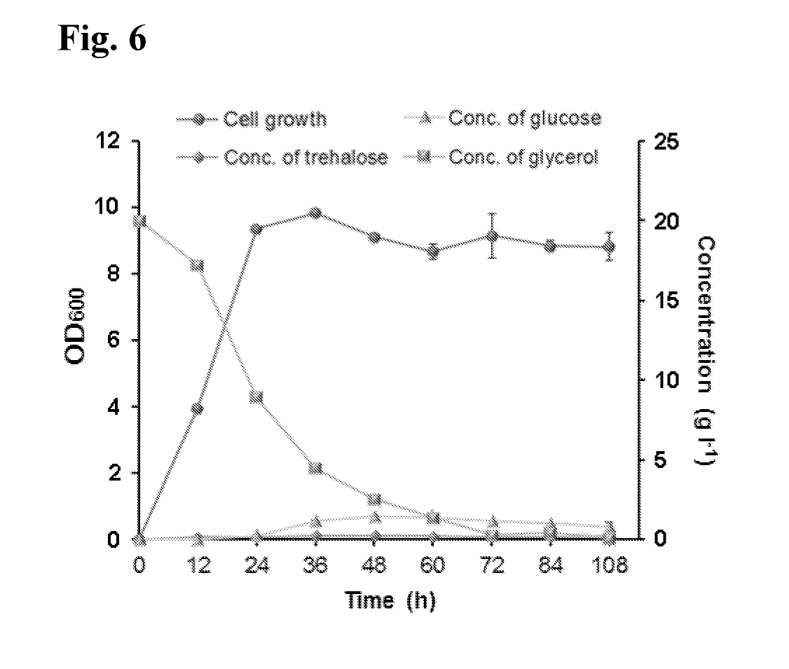

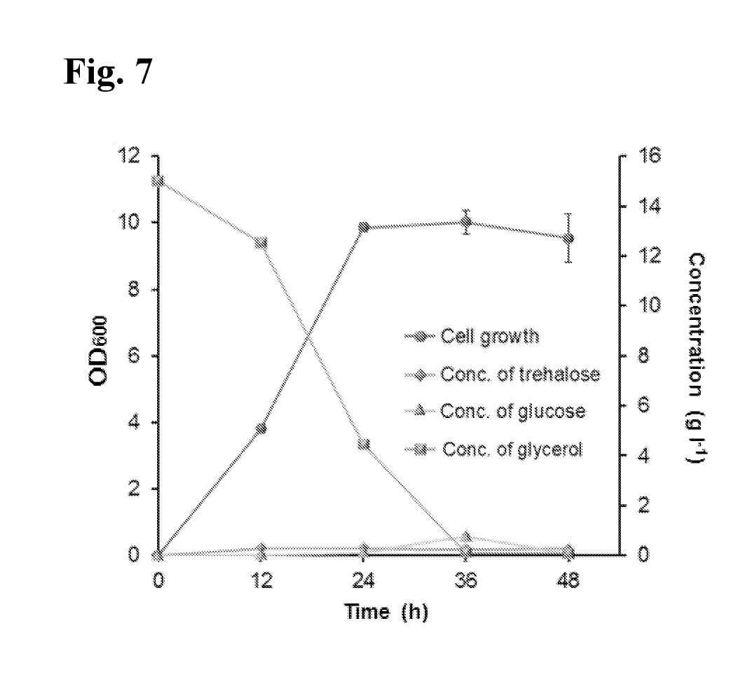

Furthermore, to test the productivity of YW-6b, we increased the initial concentration of glucose and glycerol to 15 g/L and 20 g/L, respectively. After prolonging the cultivation time to 96 h, 8.20±0.25 g/L of trehalose was accumulated by consuming 9.53±0.11 g/L glucose and 15.48±0.19 g/L glycerol in M5 medium. The final value of OD₆₀₀ was 7.05±0.61. Remarkably, the conversion efficiency of glucose to trehalose was dramatically improved to 0.86 g trehalose/g glucose reaching the theoretical maximum and the glycerol utilization efficiency were greatly improved to 0.53 g trehalose/g glycerol. Interestingly, during the later cultivation period, we noticed that the amount of produced trehalose was higher than the amount of consumed glucose, which we think was due to the gluconeogenesis. To verify this, YW-6b was cultivated in M9Y medium, which contained 20 g/L glycerol as sole carbon source. The results showed that the gluconeogenesis led to the generation of glucose from glycerol (FIG. 6). During the log phase, the glucose generation is almost negligible. However, during the stationary phase, the gluconeogenesis became more obvious and glucose was accumulated at around 1 g/L. In addition, the gluconeogenesis also led to the generation of trehalose at 0.2-0.3 g/L throughout the cultivation period. Meanwhile, we also found that the gluconeogenesis was weak in the medium with a lower concentration of glycerol (15 g/L) (FIG. 7). Hence, we concluded that the gluconeogenesis also slightly contributed to the high conversion efficiency of glucose to trehalose.

By further looking into the 96 h cultivation data, we also found that the utilization of carbon sources was different between the log phase and the stationary phase (FIG. 4D). During the log phase (12-24 h), cells grew fast and produced 2.54±0.03 g/L of trehalose by consuming 3.61±0.18 g/L glucose and 5.69±0.20 g/L glycerol. The conversion efficiency of glucose to trehalose was 0.70 g trehalose/g glucose and the glycerol utilization efficiency was 0.45 g trehalose/g glycerol. However, during stationary phase (24-96 h), cells stopped growing and continued producing 4.68±0.15 g/L more trehalose by further consuming 4.48±0.08 g/L glucose and 5.99±0.13 g/L glycerol, representing much higher carbon source conversion or utilization efficiency (1.04 g trehalose/g glucose and 0.75 g trehalose/g glycerol). On one hand, we knew the gluconeogenesis still contributed to the high conversion efficiency of glucose to trehalose especially in the stationary phase; on the other hand, the results indicated PEP can still be generated from glycerol to drive glucose uptake even when cell growth stops and the synergetic efficiency between these two carbon sources is even higher during the stationary phase because of the reduced carbon conversion into cell biomass, which are highly desired features in large-scale production.

Further Attempt on Optimization of the Synergetic Carbon Utilization Mechanism.

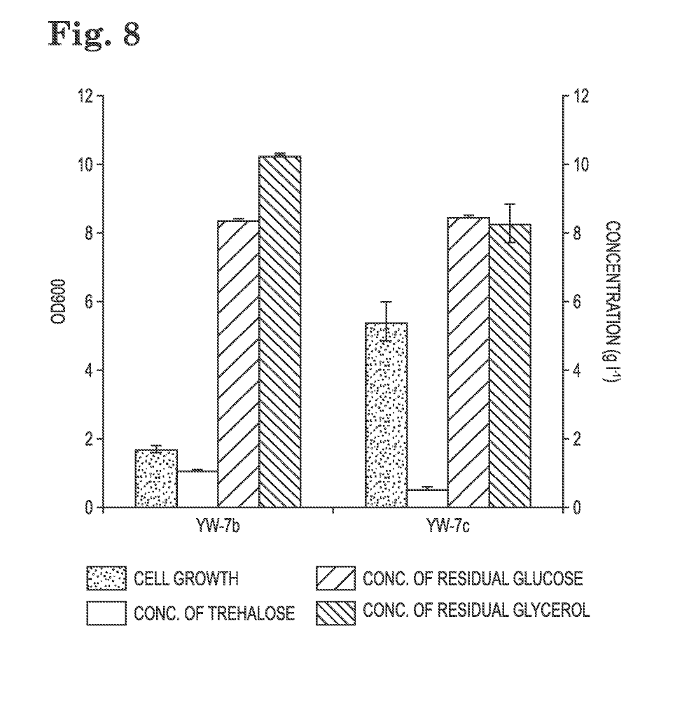

Based upon the above data and analysis, we speculated that the synergetic carbon utilization mechanism could be further optimized by reserving more PEP to drive PTS for glucose uptake. To examine this, we blocked the consumption of PEP to oxaloacetate (OAA) by deleted ppc (encoding phosphoenolpyruvate carboxylase) in strain YW-6 (FIG. 1), generating strain YW-7. This gene deletion greatly impaired cell growth. In M4 medium, YW-7 harboring pYW-1 and pYW-2 (YW-7b) only produced 1.11±0.001 g/L trehalose with an OD₆₀₀ value of 1.7±0.1 in 48 h (FIG. 8), however, the conversion efficiency of glucose to trehalose increased by 10% compared with YW-6b. Hence, we reasoned that if we could restore the cell growth, we might be able to recover the trehalose production. To verify the hypothesis, we introduced a heterologous pathway from pyruvate to OAA catalyzed by pyruvate carboxylase into YW-7b to complement the disruption of PEP conversion into OAA (FIG. 1). Gene pyc (encoding pyruvate carboxylase) from Lactococcus lactis was cloned and inserted into pYW-2 as another operon, generating plasmid pYW-3. YW-7 harboring pYW-1 and pYW-3 (YW-7c) grew better that YW-7b in M4 medium, with an OD₆₀₀ value of 5.4±0.57 (FIG. 8) in 48 h. However, the titer of trehalose was only 0.55±0.04 g/L. The consumption glucose and glycerol was 1.55±0.03 g/L and 6.35±0.16 g/L, respectively. The results indicated that deleting ppc and complementing with pyc cannot lead to further optimization of the synergetic mechanism.

Table 2 below shows a summary of the metabolic changes introduced into the various strains evaluated in this example.

TABLE 2
Selected metabolically engineered strains of E. coli

| | | | | | | | | YW- | | | | | | | |
|---|---|---|---|---|---|---|---|---|---|---|---|---|---|---|---|
| | YW- | YW- | YW- | YW- | YW- | YW- | YW- | 4b | YW- | YW- | YW- | YW- | YW- | YW- | YW- |
| | 1 | 2 | 3 | 3a | 3b | 4 | 4b | Δglk | 5 | 5b | 6 | 6b | 7 | 7b | 7c |
| ΔpgiΔzwf | X | X | | | | X | X | X | X | X | X | X | X | X | X |
| ΔpykAFΔgld | | X | | | | | | | | | X | X | X | X | X |
| A | | | | | | | | | | | | | | | |
| ΔtreACF | | | X | X | X | X | X | X | X | X | X | X | X | X | X |
| pYW-1 | | | | X | X | | X | X | | X | | X | | X | X |
| pYW-2 | | | | | X | | X | X | | X | | X | | X | |
| Δglk | | | | | | | | X | | | | | | | |
| ΔugdΔgcd | | | | | | | | | X | X | X | X | X | X | X |
| Δppc | | | | | | | | | | | | | X | X | X |
| pYW-3 | | | | | | | | | | | | | | | X |
| Trehalose g/L | | | 0.21 | 1.27 | 1.59 | | 0.24 | | | | | | | | |
| M1 (48 hr) | | | 0.07 | 0.22 | 0.07 | | 0.01 | | | | | | | | |
| Trehalose g/L | | | | | 1.22 | | 2.05* | | | 2.43 | | 3.67** | | 1.11 | 0.55 |
| M4 (48 hr) | | | | | 0.01 | | 0.48 | | | 0.08 | | 0.22 | | 0.00 | 0.04 |
| Trehalose g/L | | | | | | | | | | | | 8.20*** | | | |
| M5 (96 hr) | | | | | | | | | | | | 0.25 | | | |

*utilization of glycerol (g trehalose/g glycerol) 0.16 g/g
**utilization of glycerol (g trehalose/g glycerol) 0.29 g/g
***utilization of glycerol (g trehalose/g glycerol) 0.53 g/g; additionally, gluconeogenesis observed (glucose produced from glycerol)

### Discussion

As an enabling technology, one of the focuses of metabolic engineering is to develop microbial approaches for chemical production. The economic viability of such approaches largely depends on the utilization efficiency of carbon sources, which determines the titer, yield and productivity. Although many progresses have been made in utilizing single carbon source such as glucose or co-utilizing multiple carbon sources such as glucose and xylose for the conventional microbial synthesis that requires the breakdown of sugar molecules (Li et al., Metab. Eng. 35, 1-8 (2016); Kim et al., Metab. Eng. 30, 141-148 (2015a); Kim et al., Biotechnol. Bioeng. 112, 416-421 (2015b)), however, non-catabolic use of glucose as C6 building block or backbone precursor for microbial synthesis remains a great challenge in metabolic engineering due to the presence of strong glucose catabolism in microorganisms and its indispensable association with continuous glucose uptake, cell growth and cellular metabolism.

In this work, we proposed and developed a synergetic carbon utilization mechanism to address this challenge, which decouples glucose uptake from its catabolism by using glycerol as the carbon source to generate PEP for driving the PTS. The crosstalk and synergy between the two carbon sources glucose and glycerol were expected to be established through the glycerol-dependent PEP generation and the PTS-mediated PEP consumption. To validate the mechanism, we developed a glucose-based trehalose biosynthesis model. With successful validation and enhancement of this mechanism in the biosynthesis model, we were able to achieve efficient microbial synthesis of trehalose at over 8 g/L in shake flasks, which demonstrates great potential for large-scale production of trehalose. The conversion efficiency of glucose to trehalose reached a surprising 0.86 g trehalose/g glucose, 91% of the theoretical maximum, with the slight contribution from gluconeogenesis. The utilization efficiency of glycerol was 0.53 g trehalose/g glycerol, 28% of the theoretical maximum, which indicated the opportunities for further improvement. However, our rational attempt to reserve PEP as driving force for glucose uptake by eliminating its conversion into OAA was not successful due to its strong negative impact on cell growth, suggesting that a more systematic approach to further improve PEP intracellular availability might be necessary for further boosting the glycerol utilization efficiency.

In conclusion, this work established and demonstrated a synergetic carbon utilization mechanism for the first time. Its applicability can be potentially extended beyond the trehalose biosynthesis model demonstrated here to other microbial synthesis involving non-catabolic use of glucose as C6 building block or backbone precursor, such as chondroitin and heparin (He et al., Metab. Eng. 27, 92-100 (2015); Xu et al., Science 334, 498-501 (2011); Zhang et al., Metab. Eng. 14, 521-527 (2012); Peterson et al., Nat. Prod. Rep. 26, 610-627 (2009)). In addition, carbon sources such as xylose can also be used to replace glycerol to achieve synergetic carbon utilization, which would gain broader application when lignocellulosic hydrolysates are used as carbon sources.

### Methods

Experimental Materials.

E. coli strain XL1-Blue was used for gene cloning and preparation of plasmids. E. coli strain BW25113 was used as parent strain for generating knockout strains. Keio knockout strains were purchased from the Coli Genetic Stock Center (CGSC). E. coli strains carrying multiple gene knockouts were created by either P1 transduction or Red disruption method (Thomason et al., Curr. Protoc. Mol. Biol. Chapter 1, Unit 1 17 (2007); Doublet et al., J. Microbiol Methods 75, 359-61 (2008)). The characteristics of all the strains used in this study are described in Table 3. Luria-Bertani (LB) medium was used to grow E. coli cells for preparing plasmid and inoculum. Ampicillin and kanamycin were added to the final concentrations of 100 μg/ml and 50 μg/ml into medium, respectively, when necessary.

Plasmid Construction.

Plasmids pZE12-luc and pCS27 were used for expressing multiple enzymes involved in trehalose biosynthesis. The otsA, otsB, pgm, and galU genes were amplified from the genomic DNA of E. coli BW25113. To construct plasmid pYW1, otsA and otsB were digested with KpnI/SphI and SphI/XbaI, respectively, and then ligated with the KpnI/XbaI digested pZE12-luc fragment via three-piece ligation. To construct plasmid pYW2, pgm and galU were digested with Acc65I/SalI and SalI/BamHI, respectively, and then ligated with the Acc65I/BamHI digested pCS27 fragment. The gene of pyruvate carboxylase (PyC) from Lactococcus lactis (ATCC 19435) was cloned into pCS27 between Acc65I and BamHI, generating pCS-PyC. Plasmid pYW3 was constructed by inserting the pLlacO1-PyC operon from pCS-PyC into pYW2 using SacI and SpeI. The characteristics of the involved plasmids are described in Table 3.

TABLE 3
Strains and plasmids

| Plasmid and Strain | Characteristics | Source |
|---|---|---|
| Plasmid | | |
| pZE12-luc | ColE1 ori; Amp^{R}; pLlacO1; luc | Lin and Yan¹ |
| pCS27 | p15A ori; Kan^{R}; pLlacO1; MCS | Lin and Yan¹ |
| pCS-PyC | pCS27 vector containing the gene of PyC from Lactococcus lactis | This study |
| | (ATCC 19435) | |
| pYW1 | pZE12-luc vector containing otsA and otsB from E. coli BW25113 | This study |
| pYW2 | pCS27 vector containing pgm and galU from E. coli BW25113 | This study |
| pYW3 | pYW2 containing the pLlacO1-PyC operon from pCS-PyC | This study |
| Strain | | |
| XL1-Blue | recA1 endA1 gyrA96 thi-1 hsdR17 supE44 relA1 lac [F′ proAB | Stratagene |
| | lacIqZ ΔM15 Tn10 (TetR)] | |
| BW25113 | F−, Δ(araD-araB), ΔlacZ (::rrnB-3), λ−, rph-1, Δ(rhaD-rhaB), hsdR | Yale CGSC |
| BW25113/ΔtreA | BW25113 ΔtreA::kan | Yale CGSC |
| BW25113/ΔtreC | BW25113 ΔtreC::kan | Yale CGSC |
| BW25113/ΔtreF | BW25113 ΔtreF::kan | Yale CGSC |
| BW25113/Δpgi | BW25113 Δpgi::kan | Yale CGSC |
| BW25113/Δzwf | BW25113 Δzwf::kan | Yale CGSC |
| BW25113/Δugd | BW25113 Δugd::kan | Yale CGSC |
| BW25113/Δgcd | BW25113 Δgcd::kan | Yale CGSC |
| BW25113/ΔpykF | BW25113 ΔpykF::kan | Yale CGSC |
| YW-1 | BW25113 ΔpgiΔzwf | This study |
| YW-2 | BW25113 ΔpgiΔzwfΔpykAFΔgldA | This study |
| YW-3 | BW25113 ΔtreAΔtreCΔtreF | This study |
| YW-3a | YW-3 harboring pYW1 | This study |
| YW-3b | YW-3 harboring pYW1 and pYW2 | This study |
| YW-4 | BW25113 ΔtreAΔtreCΔtreFΔpgiΔzwf | This study |
| YW-4b | YW-4 harboring pYW1 and pYW2 | This study |
| YW-4Δglk | BW25113 ΔtreAΔtreCΔtreFΔpgiΔzwfΔglk | This study |
| YW-5 | BW25113 ΔtreAΔtreCΔtreFΔpgiΔzwfΔugdΔgcd | This study |
| YW-5b | YW-5 harboring pYW1 and pYW2 | This study |
| YW-6 | BW25113 ΔtreAΔtreCΔtreFΔpgiΔzwfΔugdΔgcdΔpykAFΔgldA | This study |
| YW-6b | YW-6 harboring pYW1 and pYW2 | This study |
| YW-7 | BW25113 ΔtreAΔtreCΔtreFΔpgiΔzwfΔugdΔgcdΔpykAFΔgldAΔppc | This study |
| YW-7b | YW-7 harboring pYW1 and pYW2 | This study |
| YW-7c | YW-7 harboring pYW1 and pYW3 | This study |

¹Lin, Y. H. & Yan, Y. J. Biosynthesis of caffeic acid in Escherichia coli using its endogenous hydroxylase complex. Microb. Cell Fact. 11(2012).

Examining Trehalose Degradation in E. coli.

Feeding experiments were conducted to examine the degradation of trehalose by several E. coli strains. E. coli strains BW25113, BW25113/ΔtreA, BW25113/ΔtreC, BW25113/ΔtreF, and YW-3 were inoculated in 3 ml LB medium and grown overnight at 37° C. Subsequently, 0.8 ml of the preinoculum was added to 20 ml of fresh M9Y medium with 5 g/L trehalose.2H₂O and grown at 37° C. with shaking (270 rpm). The M9Y medium contains 20 g/L glycerol, 5 g/L yeast extract, 1 g/L NH₄Cl, 6 g/L Na₂HPO₄, 3 g/L KH₂PO₄, 0.5 g/L NaCl, 1 mM MgSO₄, 0.1 mM CaCl₂, and 1.0 mg/L vitamin B1. Samples were taken at 12 h and 24 h, and analyzed by HPLC.

Cultivation Experiments to Test Synergetic Carbon Utilization in E. coli.

To test synergetic carbon utilization in E. coli, a series cultivation experiments were conducted in three different media with different E. coli strains. M1 medium contains 10 g/L glucose, 5 g/L yeast extract, 1 g/L NH₄Cl, 6 g/L Na₂HPO₄, 3 g/L KH₂PO₄, 0.5 g/L NaCl, 1 mM MgSO₄, 0.1 mM CaCl₂, and 1.0 mg/L vitamin B1; M2 medium was prepared by replacing 10 g/L glucose with 10 g/L glycerol; while M3 medium contains both 10 g/L glucose and 10/L glycerol as carbon sources and the other medium components remain the same. For the cultivation experiments, strain YW-1 (BW25113 ΔpgiΔzwf) was inoculated in 3 ml LB medium and grown at 37° C. for 12 h. Subsequently, 0.8 ml of the preinoculum was re-inoculated into 20 ml of above three different media and grown at 37° C. with shaking (270 rpm) for 56 h, respectively. In parallel, E. coli BW25113 was used as control strain. In addition, strain YW-2 (BW25113 ΔpgiΔzwfΔpykAFΔgldA) was also employed for the cultivation experiments in M3 medium. The cultivation procedure and condition are the same. For all the above cultivation experiments, samples were taken every 4 hours from 0 h to 16 h and every 8 hours from 16 h to 56 h. OD₆₀₀ values were measure. The consumption of glucose and glycerol was analyzed by HPLC.

Microbial Synthesis of Trehalose in E. coli.

To examine the applicability of the synergetic carbon utilization mechanism and further enhance its efficiency, a series of shake flask experiments for microbial synthesis of trehalose were conducted in three different media by using different E. coli strains. The above M1 medium was still used for the shake flask experiments. In addition, M4 and M5 media were also used, which contains 10 g/L glucose plus 15 g/L glycerol and 15 g/L glucose plus 20 g/L glycerol, respectively. The other components of M4 and M5 medium are the same as those of M1 medium.

To construct the trehalose biosynthesis model, strains YW-3 (BW25113 ΔtreAΔtreCΔtreF), YW-3a (YW-3 carrying plasmid pYW-1), and YW-3b (YW-3 carrying plasmids pYW-1 were pYW-2) were inoculated in 3 ml LB medium and grown at 37° C. for 8 h, respectively. Subsequently, 0.8 ml of each preinoculum was re-inoculated into 20 ml of M1 medium and grown at 30° C. with shaking (270 rpm) for 48 h. Samples were taken at 48 h. We used the trehalose biosynthesis model to validate the synergetic carbon utilization mechanism. For this purpose, Plasmids pYW-1 and pYW-2 was introduced into YW-4 (BW25113 ΔtreAΔtreCΔtreFΔpgiΔzwf) to generate YW-4b. YW-4b was inoculated in 3 ml LB medium and grown at 37° C. for 8 h, then 0.8 ml of the preinoculum was re-inoculated into 20 ml of M4 medium and grown at 30° C. with shaking (270 rpm) for 48 h. Samples were taken every 8 hours. As control, M1 medium was used to grow strain YW-4b for trehalose biosynthesis. In addition, YW-4Δglk harboring pYW-1 and pYW-2 was also used as control to evaluate the contribution of glucokinase to trehalose biosynthesis in M4 medium. To further improve the synergetic carbon utilization efficiency, strains YW-5b, YW-6b, YW-7b, and YW-7c were also generated as in Table 3 and used for trehalose biosynthesis in M4 medium as strain YW-4b. Samples were taken either at 48 h or every 8 hours. To optimize trehalose production, M5 medium was used to cultivate strain YW-6b for 96 hours with shaking (300 rpm). The inoculation procedure was the same as that of YW-4b. Samples were taken every 12 hours. For all the above cultivation, we added IPTG to the cultures with a final concentration of 0.5 mM at the beginning. For all the samples, OD₆₀₀ values were measured and HPLC analysis was conducted.

Evaluating gluconeogenesis in E. coli.

To evaluate gluconeogenesis, we used M9Y and M9Y-1 media and strain YW-6b. The components of M9Y and M9Y-1 media are the same expect for glycerol, which is 20 g/L in M9Y and 15 g/L in M9Y-1, respectively. YW-6b was inoculated in 3 ml LB medium and grown at 37° C. for 8 h. Subsequently, 0.8 ml of the preinoculum was re-inoculated into 20 ml of M9Y medium and M9Y-1 medium, respectively. For YW-6b in M9Y medium, it was grown at 30° C. with shaking (300 rpm) for 108 h. For YW-6b in M9Y-1 medium, it was grown at 30° C. with shaking (270 rpm) for 48 h. Samples were taken every 12 hours. IPTG with the final concentration of 0.5 mM was added to the cultures at the beginning. For each sample, OD₆₀₀ values were measured and HPLC analysis was conducted.

HPLC-RID Analysis.

The analysis of the samples collected above was done by HPLC (Shimadzu) equipped with a Coregel-64H column (Transgenomic). Samples (1 ml) were centrifuged at 15,000 rpm for 10 minutes. The supernatants were filtered and used for analysis. The mobile phase used was 20 mN H₂SO₄ having a flow rate of 0.6 ml/min. The oven temperature set at 40° C. (Eiteman et al., Anal. Chim. Acta 338, 69-75 (1997)).

### REFERENCES

1. Munoz-Elias, E. J. & McKinney, J. D. Carbon metabolism of intracellular bacteria. Cell. Microbiol. 8, 10-22 (2006).
2. Lin, Y. H., Shen, X. L., Yuan, Q. P. & Yan, Y. J. Microbial biosynthesis of the anticoagulant precursor 4-hydroxycoumarin. Nat. Commun. 4 (2013).
3. Lin, Y. H., Sun, X. X., Yuan, Q. P. & Yan, Y. J. Engineering bacterial phenylalanine 4-hydroxylase for microbial synthesis of human neurotransmitter precursor 5-hydroxytryptophan. ACS Synth. Biol. 3, 497-505 (2014).
4. Lin, Y. H., Sun, X. X., Yuan, Q. P. & Yan, Y. J. Extending shikimate pathway for the production of muconic acid and its precursor salicylic acid in Escherichia coli. Metab. Eng. 23, 62-69 (2014).
5. Yuzawa, S., Chiba, N., Katz, L. & Keasling, J. D. Construction of a part of a 3-hydroxypropionate cycle for heterologous polyketide biosynthesis in Escherichia coli. Biochemistry 51, 9779-9781 (2012).
6. Sun, X. X., Lin, Y. H., Huang, Q., Yuan, Q. P. & Yan, Y. J. A novel muconic acid biosynthesis approach by shunting tryptophan biosynthesis via anthranilate. Appl. Environ. Microbiol. 79, 4024-4030 (2013).
7. Lin, Y. H., Sun, X. X., Yuan, Q. P. & Yan, Y. J. Combinatorial biosynthesis of plant-specific coumarins in bacteria. Metab. Eng. 18, 69-77 (2013).
8. Peralta-Yahya, P. P., Zhang, F. Z., Del Cardayre, S. B. & Keasling, J. D. Microbial engineering for the production of advanced biofuels. Nature 488, 320-328 (2012).
9. Santos, C. N. S., Koffas, M. & Stephanopoulos, G. Optimization of a heterologous pathway for the production of flavonoids from glucose. Metab. Eng. 13, 392-400 (2011).
10. Atsumi, S., Hanai, T. & Liao, J. C. Non-fermentative pathways for synthesis of branched-chain higher alcohols as biofuels. Nature 451, 86-U13 (2008).
11. Stephanopoulos, G. Challenges in engineering microbes for biofuels production. Science 315, 801-804 (2007).
12. Farmer, W. R. & Liao, J. C. Improving lycopene production in Escherichia coli by engineering metabolic control. Nat. Biotechnol. 18, 533-537 (2000).
13. Lim, C. G. et al. Development of a recombinant Escherichia coli strain for overproduction of the plant pigment anthocyanin. Appl. Environ. Microbiol. 81, 6276-6284 (2015).
14. Wang, S. Y. et al. Efficient glycosylation of puerarin by an organic solvent-tolerant strain of Lysinibacillus fusiformis. Enzyme Microb. Technol. 57, 42-47 (2014).
15. Yan, Y., Chemler, J., Huang, L., Martens, S. & Koffas, M. A. Metabolic engineering of anthocyanin biosynthesis in Escherichia coli. Appl. Environ. Microbiol. 71, 3617-3623 (2005).
16. Shiue, E., Brockman, I. M. & Prather, K. L. J. Improving product yields on D-glucose in Escherichia coli via knockout of pgi and zwf and feeding of supplemental carbon sources. Biotechnol. Bioeng. 112, 579-587 (2015).
17. Pandey, R. P., Malla, S., Simkhada, D., Kim, B. G. & Sohng, J. K. Production of 3-O-xylosyl quercetin in Escherichia coli. Appl. Microbiol. Biotechnol. 97, 1889-1901 (2013).
18. Hernandez-Montalvo, V. et al. Expression of galP and glk in a Escherichia coli PTS mutant restores glucose transport and increases glycolytic flux to fermentation products. Biotechnol. Bioeng. 83, 687-694 (2003).
19. Schiraldi, C., Di Lernia, I. & De Rosa, M. Trehalose production: exploiting novel approaches. Trends Biotechnol. 20, 420-425 (2002).
20. Ohtake, S. & Wang, Y. J. Trehalose: Current use and future applications. J. Pharm. Sci. 100, 2020-2053 (2011).
21. Kidd, G. & Devorak, J. Trehalose is a sweet target for agbiotech. Nat Biotech. 12, 1328-1329 (1994).
22. Patist, A. & Zoerb, H. Preservation mechanisms of trehalose in food and biosystems. Colloids Surf B Biointerfaces 40, 107-113 (2005).
23. Kim, Y. C., Quan, F. S., Compans, R. W., Kang, S. M. & Prausnitz, M. R. Formulation and coating of microneedles with inactivated influenza virus to improve vaccine stability and immunogenicity. J. Control Release 142, 187-195 (2010).
24. Torrice, M. Insect sugar could treat fatty liver disease. Chem. Eng. News 94, 9-9 (2016).
25. Global Trehalose Market Size. (http://globalqyresearch.com/press-releases/global-trehalose-market)
26. Kobayashi, K., Komeda, T., Miura, Y., Kettoku, M. & Kato, M. Production of trehalose from starch by novel trehalose-producing enzymes from Sulfolobus solfataricus KM1. J. Ferment. Bioeng. 83, 296-298 (1997).
27. Mukai, K. et al. Production of trehalose from starch by thermostable enzymes from Sulfolobus acidocaldarius. Starch-Starke 49, 26-30 (1997).
28. Yoshida, M., Nakamura, N. & Horikoshi, K. Production of trehalase from starch by maltose phosphorylase and trehalose phosphorylase from a strain of plesiomonas. Starch-Starke 49, 21-26 (1997).
29. Koh, S., Shin, H. J., Kim, J. S., Lee, D. S. & Lee, S. Y. Trehalose synthesis from maltose by a thermostable trehalose synthase from Thermos caldophilus. Biotechnol. Lett. 20, 757-761 (1998).
30. Koh, S. et al. Mechanistic study of the intramolecular conversion of maltose to trehalose by Thermus caldophilus GK24 trehalose synthase. Carbohydr. Res. 338, 1339-1343 (2003).
31. Deutscher, J. The mechanisms of carbon catabolite repression in bacteria. Curr. Opin. Microbiol. 11, 87-93 (2008).
32. Steinsiek, S. & Bettenbrock, K. Glucose transport in Escherichia coli mutant strains with defects in sugar transport systems. J. Bacteriol. 194, 5897-5908 (2012).
33. Morita, T., El-Kazzaz, W., Tanaka, Y., Inada, T. & Aiba, H. Accumulation of glucose 6-phosphate or fructose 6-phosphate is responsible for destabilization of glucose transporter mRNA in Escherichia coli. J. Biol. Chem. 278, 15608-15614 (2003).
34. Kandror, O., DeLeon, A. & Goldberg, A. L. Trehalose synthesis is induced upon exposure of Escherichia coli to cold and is essential for viability at low temperatures. Proc. Natl. Acad. Sci. USA 99, 9727-9732 (2002).
35. Strom, A. R. & Kaasen, I. Trehalose Metabolism in Escherichia coli: stress protection and stress regulation of gene expression. Mol. Microbiol. 8, 205-210 (1993).
36. Li, Z. J. et al. Biosynthesis of poly(glycolate-co-lactate-co-3-hydroxybutyrate) from glucose by metabolically engineered Escherichia coli. Metab. Eng. 35, 1-8 (2016).
37. Kim, S. M. et al. Simultaneous utilization of glucose and xylose via novel mechanisms in engineered Escherichia coli. Metab. Eng. 30, 141-148 (2015).
38. Kim, S. Y., Lee, J. & Lee, S. Y. Metabolic engineering of Corynebacterium glutamicum for the production of L-ornithine. Biotechnol. Bioeng. 112, 416-421 (2015).
39. He, W. Q. et al. Production of chondroitin in metabolically engineered E. coli. Metab. Eng. 27, 92-100 (2015).
40. Xu, Y. M. et al. Chemoenzymatic synthesis of homogeneous ultralow molecular weight heparins. Science 334, 498-501 (2011).
41. Zhang, C. Y. et al. Metabolic engineering of Escherichia coli BL21 for biosynthesis of heparosan, a bioengineered heparin precursor. Metab. Eng. 14, 521-527 (2012).
42. Peterson, S., Frick, A. & Liu, J. Design of biologically active heparan sulfate and heparin using an enzyme-based approach. Nat. Prod. Rep. 26, 610-627 (2009).
43. Thomason, L. C., Costantino, N. & Court, D. L. E. coli genome manipulation by P1 transduction. Curr. Protoc. Mol. Biol. Chapter 1, Unit 1 17 (2007).
44. Doublet, B. et al. Antibiotic marker modifications of lambda Red and FLP helper plasmids, pKD46 and pCP20, for inactivation of chromosomal genes using PCR products in multidrug-resistant strains. J. Microbiol Methods 75, 359-61 (2008).
45. Eiteman, M. A. & Chastain, M. J. Optimization of the ion-exchange analysis of organic acids from fermentation. Anal. Chim. Acta 338, 69-75 (1997).

The foregoing summary, description of illustrative embodiments and examples have been given for clarity of understanding only. No unnecessary limitations are to be understood therefrom. The invention is not limited to the exact details shown and described, for variations obvious to one skilled in the art will be included within the invention defined by the claims.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The complete disclosures of all patents, patent applications including provisional patent applications, publications including patent publications and nonpatent publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference.

1. A genetically engineered microbe comprising:
at least one metabolic pathway modification that disrupts glucose catabolism; and
at least one metabolic pathway modification that metabolically redirects phosphoenolpyruvate (PEP) for enhanced uptake of glucose.

2. The genetically engineered microbe of claim 1, wherein the metabolic pathway modification that disrupts glucose catabolism comprises a modification of the glycolysis pathway, or a modification in the pentose phosphate pathway, or both.

3. The genetically engineered microbe of claim 1, wherein the metabolic pathway modification that metabolically redirects PEP comprises a modification that disrupts a PEP-dependent glycerol assimilation pathway.

4. The genetically engineered microbe of claim 1, further comprising at least one metabolic pathway modification selected from the group consisting of:
(a) a metabolic pathway modification that disrupts the conversion of UDP-glucose to UDP glucuronic acid;
(b) a metabolic pathway modification that eliminates the conversion of glucose-1-phosphate to glucolactone;
(c) a metabolic pathway modification that enhances the UDP-glucose biosynthetic pathway so as to direct more glucose into UDP-glucose;
(d) a metabolic pathway modification that enhances the consumption or conversion of glucose-6-phosphate;
(e) a metabolic pathway modification that disrupts a metabolic pathway associated with degradation of a glycosylated compound or a metabolic pathway that diverts a precursor away from the glycosylated compound; and
(f) a metabolic pathway modification that disrupts a metabolic pathway associated with degradation of a glycosylated compound or a metabolic pathway that diverts a precursor away from the glycosylated compound.

5.-9. (canceled)

10. The genetically engineered microbe of claim 1, which simultaneously utilizes glucose and a secondary sugar as carbon sources.

11. The genetically engineered microbe of claim 10, wherein the secondary sugar comprises at least one of glycerol or xylose.

12. (canceled)

13. The genetically engineered microbe of claim 10, wherein phosphoenolpyruvate (PEP) generated from consumption of the secondary sugar is utilized by the phosphotransferase system (PTS) so as to drive glucose uptake for production of a glycosylated compound.

14. The genetically engineered microbe of claim 1 comprising a synergetic carbon utilization mechanism that
(a) decouples glucose uptake from glucose catabolism by using glycerol as a carbon source to generate phosphoenolpyruvate (PEP) for operating the phosphotransferase system; or
(b) couples glucose uptake with glycerol catabolism via the phosphoenolpyruvate (PEP) as a driving force for glucose transport;
or both (a) and (b).

15.-17. (canceled)

18. The genetically engineered microbe of claim 1, comprising at least one mutation selected from the group consisting of Δpgi, Δzwf, ΔpykA, ΔpykF, ΔgldA, Δugd, and Δgcd (E. coli) or their counterparts in other microbes.

19. The genetically engineered microbe of claim 1, wherein the microbe expresses or overexpresses at least one enzyme encoded by galU or pgm (E. coli) or counterparts in other microbes.

20. The genetically engineered microbe of claim 1, wherein the microbe produces trehalose, and wherein the microbe further comprises at least one mutation selected from the consisting of ΔtreA, ΔtreC, and ΔtreF (E. coli) or counterparts in other microbes.

21. The genetically engineered microbe of claim 1, wherein the microbe expresses or overexpresses at least one enzyme encoded by otsA or otsB (E. coli) or counterparts in other microbes.

22. The genetically engineered microbe of claim 1, comprising an E. coli cell comprising at least one deletion mutation selected from the group consisting of
(a) ΔpgiΔzwf,
(b) ΔpykAFΔgldA;
(c) ΔtreACF;
(d) Δglk;
(e) ΔugdΔgcd;
(f) Δppc; and
(g) any combination thereof.

23. The genetically engineered microbe of claim 22, comprising an E. coli cell comprising at least one plasmid expressing at least one enzyme operably encoded by at least one member of the group consisting of otsA, otsB, pgm, and galU.

24.-26. (canceled)

27. The genetically engineered E. coli cell of claim 22, which is further metabolically engineered to enhance expression of phosphoglucomutase (pgm) or UTP-glucose-1-phosphate uridylyltransferase) (galU) or both.

28. (canceled)

29. A method for producing a glycosylated compound comprising culturing the microbe of claim 1 under conditions to produce the glycosylated compound.

30. The method of claim 29, wherein the glycosylated compound is selected from the group consisting of a glycoprotein, glycopeptide, glycolipid, proteoglycan, antibody, glycan, glycoside, polysaccharide, nucleotide and nucleic acid.

31. The method of claim 30, wherein the polysaccharide comprises trehalose, chondroitin or heparin.

32.-33. (canceled)

34. The method of claim 30, wherein glucose and at least one of glycerol or xylose are supplied as carbon sources.

35.-37. (canceled)

38. The genetically engineered microbe of claim 1, wherein the microbe is a bacterial cell or a yeast cell.

39.-40. (canceled)
